(19) United States
(12) Patent Application Publication
(10) Pub. No.: US 2015/0104426 A1
(43) Pub. Date: Apr. 16, 2015

(54) SKIN-CARE AGENT CONTAINING PLEUROTUS FERULAE FRUIT BODY EXTRACT OR PLEUROTUS FERULAE MYCELIUM EXTRACT

(71) Applicant: AMI Cosmetic Co., Ltd., Seoul (KR)

(72) Inventors: Kyung Rok Lee, Seoul (KR); Il Hong, Seoul (KR); Do Gyeong Lee, Gyeongsan (KR); Jung No Lee, Cheongju (KR); Soo Yeon Choi, Daejeon (KR); Nu Rim Lee, Sinan-gun (KR)

(73) Assignee: AMI Cosmetic Co., Ltd., Seoul (KR)

(21) Appl. No.: 14/079,129

(22) Filed: Jan. 27, 2014

(30) Foreign Application Priority Data
Oct. 11, 2013 (KR) 10-2013-0120983

(51) Int. Cl. A61K 36/07; A61Q 19/02; A61Q 7/00; A61Q 19/00; A61Q 5/00; A61K 8/96; A61Q 19/08

(52) U.S. Cl. CPC A61K 36/07; A61K 8/96; A61Q 19/02; A61Q 19/08; A61Q 19/007; A61Q 5/00; A61Q 7/00
USPC 424/93.5; 424/195.15

(57) ABSTRACT

Disclosed is a composition for skin external application containing a Pleurotus ferulae fruiting body extract, a Pleurotus ferulae mycelium extract or a liquid culture obtained by culturing the Pleurotus ferulae mycelium. More specifically, disclosed is a functional composition for skin external application containing 0.001 to 90.0% by weight of a Pleurotus ferulae fruiting body extract, a Pleurotus ferulae mycelium extract or a liquid culture obtained by culturing the Pleurotus ferulae mycelium, with respect to the weight of the composition, wherein the composition is potently effective in scavenging active oxygen, preventing aging, acting wrinkles, facilitating collagen synthesis, moisturizing skin, whitening skin, reducing skin irritation, preventing acne, alleviating atopy, providing anti-inflammation, preventing hair damage, preventing alopecia and improving hair growth.

### BACKGROUND OF THE INVENTION

1. Field of the Invention

The present invention relates to a composition for skin external application containing a Pleurotus ferulae fruiting body extract, a Pleurotus ferulae mycelium extract or a liquid culture obtained by culturing the Pleurotus ferulae mycelium (hereinafter, referred to simply as “Pleurotus ferulae mycelium culture”).

2. Description of the Related Art

Aging of the skin is classified into two types, i.e., intrinsic (chronological) aging and photoaging [Gilchrest B A: J. Am. Acad. Dermatol., 21, 610-613 (1989)]. Intrinsic aging naturally occurs due to decrease in physiologic functions with age [Braverman I M, et al.: J. Invest. Dermatol., 78, 434-443 (1982)]. Photoaging means change associated with appearance and functions of the skin, caused by repeated exposure of the skin to solar radiation [Ridder G M et al.: J. Am. Acad. Dermatol., 25, 751-760 (1991)]. In addition, aging of the skin may be caused by ultraviolet radiation, stress, disease conditions, environmental factors, wounds and activation of active oxygen species with age. As such conditions worsen, antioxidant defense mechanisms present in vivo are destructed, cells and tissues are damaged, and adult diseases and aging are facilitated. More specifically, oxidation of lipids, proteins, polysaccharides, nucleic acids and the like, which are primary components of the skin, and destruction of skin cells and tissues thus causes aging of the skin. In particular, oxidation of proteins involves cleavage of collagen, hyaluronic acid, elastin, proteoglycan, fibronetin and the like, which are connective tissues of the skin, results in over-inflammation, causes damage to elasticity of the skin and, in serious cases, brings about mutations caused by DNA modification, emergence of cancers and deterioration in immune functions.

Accordingly, it is necessary to protect the skin through scavenge of active oxygen species produced during in vivo metabolic processes or active oxygen species mediated by ultraviolet irradiation and inflammation, and to degenerate and proliferate damaged cells through active metabolism so as to restore the skin and keep the skin healthy. An enzyme called “matrix metalloproteinase (MMP)” as well as active oxygen species is mediated in aging. Synthesis and decomposition of the extracellular matrix such as collagen are suitably controlled in vivo, but the synthesis is deteriorated with aging, expression of collagenase, i.e., matrix metalloproteinase (MMP) is facilitated, elasticity of the skin is deteriorated and wrinkles are created. In addition, exposure to ultraviolet radiation may cause activation of such a decomposition enzyme. Thus, there is a demand for development of substances which control expression of MMP activated in cells by ultraviolet radiation or inhibit the activity of MMP. Most ingredients used as raw materials of cosmetics to date simply inhibit only activity of matrix metalloproteinases (MMPs).

Meanwhile, melanin is produced through conversion of tyrosine into dopa, dopaquinone and then dopachrome by actions of tyrosinase present in pigment cells. Melanin is present in the skin, which protects the body from ultraviolet radiation and has an essential function on control of hormone secretion in vivo. However, overproduced melanin is known to create spots, freckles and the like, accelerate aging of the skin and play an important role to induce skin cancers. As such, research and development to prevent melanin over-production is actively underway. Ascorbic acid (Japanese Patent Publication Sho. 4-9320), hydroquinone (Japanese Patent Publication Sho. 6-192062), kojic acid (Japanese Patent Publication Hei. 56-7710), arbutin (Japanese Patent Publication Sho. 4-93150), plant extracts and the like have already been used for whitening cosmetics owing to inhibitory activity against tyrosinase, but the use thereof is not limited due to problems such as decomposition and discoloration caused by bad stability in cosmetic formulations, generation of off-flavor, unclear in vivo efficacies and effects, and safety.

Meanwhile, atopic dermatitis is a representative disease of patients with atopic allergies. The cause of atopic dermatitis is not clearly known, but atopic dermatitis is thought to be an immune disease in spite of being a disease caused by genetic factors, because 70% of atopic dermatitis patients have atopic history, and atopic dermatitis manifests in allergic persons, in particular, involves other allergic diseases such as allergic rhinitis and asthma.

Drugs such as adrenocortical hormones having the potential of excellent medicinal effects are used for the treatment of atopic dermatitis, but there are problems associated with side effects of adrenocortical hormone drugs. A rebound phenomenon (wherein the severity of symptoms becomes extremely worse when medication is discontinued) is well known to occur upon discontinuation of adrenocortical hormone drugs. As measures to prevent such a side effect, nonsteroidal anti-inflammatory drugs, antihistaminic agents and the like which do not contain hormones such as adrenocortical hormones are used, but these drugs do not completely treat atopic dermatitis. For this reason, there is an urgent demand for development of drugs free of side-effects for the alleviation or treatment of atopic dermatitis.

In recent years, much attention is focused on functional cosmetics having functions such as antioxidative, anti-wrinkling, whitening and anti-itching functions, produced from natural extracts in order to reduce skin irritation caused by various chemicals. For example, U.S. Pat. No. 5,972,341 discloses wrinkle alleviation effects of Commiphora mukul extracts, Japanese Patent Publication Sho. 9-672662 discloses seaweed extracts having inhibitory efficacy against hyaluronidase, Japanese Patent Publication Sho. 10-85905 discloses skin texture improvement effect of fucoidan extracted from Wakame, Japanese Patent Publication Sho. 2-245087 discloses antioxidative effects of Sargassum extracts, Korean Patent No. 0431076 discloses a cosmetic composition for treating and alleviating atopic skin, containing blettia rhizome, perilla, Echinacea and fermented soybean extracts, and Korean Patent No. 0511494 discloses extracts and compositions for treating atopic dermatitis containing Pleuropterus multiflorus TURCZ, Diospyros kaki Thunb and illite.

In addition, male pattern alopecia is dependent upon male hormones and is thus directly related to amounts of male hormones. For this reason, a great deal of research associated with the prevention and treatment of alopecia through inhibition of male hormone activities has recently been reported. Meanwhile, when functions of sebaceous glands become active due to increased male hormone secretion, overproduced sebum is congested in hair follicles due to hyperkeratinization of the hair follicle wall and comedones are created. These comedones are an initial stage of acne. Mechanisms of emergence of alopecia and acne via male hormones are as follows. 5-α-reductase is a male hormone present in male hormone-reactive tissues such as sebaceous glands, hair follicles, prostate and epididymis, and is an enzyme mediating in metabolism of testosterone into dihydrotestosterone, wherein the metabolism requires NADPH. In addition, testosterone mediates in tissues corresponding to male sexual drive, increase in skeletal muscles, male external genitalia, scrotal growth, spermatogenesis, etc., while dihydrotestosterone mediates in tissues corresponding to acne, increase in sebum alopecia, prostatic hypertrophy, etc.

In particular, over-secretion of male hormones after adolescence induces acne and alopecia. In this regard, research is actively underway to develop drugs for preventing alopecia or acne using 5-α-reductase inhibitors to prevent over-production of dihydrotestosterone which is an active type of male hormones produced by 5-α-reductase.

In an attempt to solve these problems associated with the skin, a variety of cosmetics employing natural substances or medicinally edible mushroom extracts have been developed for reduction of skin irritation by various chemical substances. Cosmetic compositions employing mushroom extracts known to date include cosmetic compositions employing mushroom extracts of Paecilomyces japonica (Korean Patent No. 0340185), Phellinus linteus, Schizophyllum commun (Korean Patent Nos. 0681703 and 0295623), Grifola frondosa (Korean Patent No. 0438009), Lentinus edode and the like. Research has been continuously made on effects of these extracts on skin anti-aging.

Examples and kinds of mushrooms used in medicinal and edible applications are innumerable, but only some of mushrooms have been used and researched for cosmetic compositions.

Meanwhile, Pleurotus ferulae is the name given to grow in glassroots called “ferula asafetida” in China. Pleurotus ferulae is rich in vitamins A, E, D₃, etc., contains about two-fold higher proteins than Pleurotus estreatus and is suitable for diet foods owing to high dietary fiber content.

In addition, Pleurotus ferulae contains about six-fold more vitamins than Pleurotus eryngii Quel, a great amount of linoleic acid as unsaturated fatty acid, and high levels of zinc and iron, thus being effective in the prevention of adult diseases as well as aging.

Whether or not Pleurotus ferulae has the effects associated with skin external agent on skin anti-aging, whitening, skin irritation reduction, atopy alleviation, acne alleviation, hair damage prevention, alopecia prevention, hair growth improvement and inflammatory disease prevention or treatment has yet to be demonstrated at all.

### RELATED ART

### Patent Document

(Patent document 1) Korean Patent No. 10-0438009 (entitled “cosmetic composition containing Grifola frondosa mycelium extract”) discloses a cosmetic composition containing a Grifola frondosa mycelium or an extract obtained from a liquid culture thereof, wherein the cosmetic composition is effective in exhibition of anti-oxidation, facilitation of skin cell proliferation and collagen production, and inhibition of production of melanin in the skin, but this patent does not disclose use of Pleurotus ferulae fruiting body extracts or Pleurotus ferulae mycelium cultures.
(Patent document 2) Korean Patent No. 0340185 (entitled “anti-wrinkle cosmetic composition containing Paecilomyces japonica extract”) discloses a cosmetic composition which effectively prevents wrinkles, based on Paecilomyces japonica extract, but this patent does not disclose use of Pleurotus ferulae fruiting body extracts or Pleurotus ferulae mycelium cultures.

### SUMMARY OF THE INVENTION

Therefore, the present invention has been made in view of the above problems, and it is one object of the present invention to provide a composition for skin external application containing natural extracts which is effective in providing anti-oxidation, promoting collagen synthesis, alleviating skin fine wrinkle, providing whitening and moisturizing, reducing skin irritation, preventing acne, alleviating atopy, providing anti-inflammation, preventing hair damage, preventing alopecia and improving hair growth.

It is another object of the present invention to provide a composition for skin external application containing, as an active ingredient, a Pleurotus ferulae (higher fungi) fruiting body extract, a Pleurotus ferulae mycelium extract or a Pleurotus ferulae mycelium culture.

It is another object of the present invention to provide a method for efficiently obtaining a Pleurotus ferulae fruiting body extract or a Pleurotus ferulae mycelium extract which has multiple effects of anti-oxidation, collagen synthesis facilitation, skin fine wrinkle alleviation, whitening, moisturizing, skin irritation reduction, acne prevention, atopy alleviation, anti-inflammation, hair damage prevention, alopecia prevention and hair growth.

In accordance with the present invention, the above and other objects can be accomplished by the provision of a composition for skin external application containing, as an active ingredient, a Pleurotus ferulae fruiting body extract, a Pleurotus ferulae mycelium extract or a Pleurotus ferulae mycelium culture.

### DETAILED DESCRIPTION OF THE INVENTION

Hereinafter, embodiments of the prevent invention will be described in detail.

The composition for external preparation according to the present invention contains a Pleurotus ferulae fruiting body extract, a Pleurotus ferulae mycelium extract or a Pleurotus ferulae mycelium culture as an active ingredient, so as to provide a composition for skin external application which is potently effective in anti-oxidation, anti-aging, wrinkle alleviation, skin moisturizing, promotion of collagen synthesis, whitening, alleviation, prevention or treatment of atopic dermatitis, reduction of skin irritation, prevention or treatment of inflammatory diseases, alleviation, prevention or treatment of acne, prevention of hair damage, prevention of alopecia and improvement of hair growth.

The Pleurotus ferulae fruiting body extract of the present invention is obtained by extracting the Pleurotus ferulae fruiting body. There is no limitation as to the type of the Pleurotus ferulae fruiting body. That is, the Pleurotus ferulae fruiting body may be naturally derived or artificially cultured.

In addition, the extraction may be carried out using a method commonly used in the technical field to which the present invention pertains. For example, a fruiting body of Pleurotus ferulae is lyophilized and ground, and the resulting fruiting body powder is extracted at a temperature of 10° C. to 30° C. in an about 2- to about 20-fold amount of, preferably, in an about 2 to about 5-fold amount of a polar solvent such as water, a lower (C₁-C₄) alcohol such as methanol or ethanol or a mixture thereof, using a method such as cold-immersion extraction, reflux cold extraction, hot water extraction, ultrasonic extraction or ultrahigh-pressure extraction.

In addition, preferably, the extract thus obtained is filtered, concentrated under reduced pressure or lyophilized and is then dissolved in the polar solvent. For example, the obtained extract is filtered, concentrated under reduced pressure in a concentrator at a temperature of 50° C. or less and lyophilized, and a target extract containing 0.001 to 70.0% by weight of the resulting substance is prepared using at least one solvent selected from purified water, ethanol, butylene glycol and propylene glycol.

In addition, the Pleurotus ferulae fruiting body extract of the present invention may mean polysaccharides contained as an active ingredient in the extract thus obtained.

Meanwhile, the Pleurotus ferulae mycelium extract of the present invention is obtained by extracting a Pleurotus ferulae mycelium. The extraction may be carried out using a method commonly used in the technical field to which the present invention pertains. For example, the Pleurotus ferulae mycelium extract may be a hot-water extract of a mycelium powder obtained by lyophilizing and grinding the Pleurotus ferulae mycelium, or an organic solvent extract obtained by extraction using methanol, ethanol, butanol or a mixture thereof.

Meanwhile, the liquid culture obtained by culturing the Pleurotus ferulae mycelium, that is, Pleurotus ferulae mycelium culture may be used per se, or as a powder obtained by drying or lyophilizing. Preferably, the liquid culture may be filtered prior to use. Specifically, the Pleurotus ferulae mycelium is liquid-cultured, the mycelium is removed by centrifugation, the culture residue is filtered through a paper filter and the resulting filtrate is then used as the liquid culture. The culture includes meaning of fermentation.

In addition, the Pleurotus ferulae mycelium extract or liquid culture obtained by culturing the Pleurotus ferulae mycelium according to the present invention may be the hot-water extract, the organic solvent extract or polysaccharides extracted from the filtrate.

In addition, the Pleurotus ferulae mycelium extract or the Pleurotus ferulae mycelium culture according to the present invention is preferably a Pleurotus ferulae mycelium extract containing one or more types of animal, plant or herb medicinal ingredients as substrates. The animal, plant or herb medicinal ingredients are not particularly limited and those commonly used in the technical field to which the present invention pertains may be used as substrates.

The Pleurotus ferulae fruiting body extract, the Pleurotus ferulae mycelium extract or the Pleurotus ferulae mycelium culture is potently effective in anti-oxidative action, inhibition of matrix metalloproteinase-1 (MMP-1) activity, inhibition of expression of matrix metalloproteinase-1 (MMP-1), facilitation of procollagen biosynthesis, facilitation of elastin production, whitening, inhibition of inflammation-inducing enzyme (hyaluronidase) activity, reduction of cytotoxicity caused by ultraviolet irradiation, inhibition of expression of inflammatory cytokine by ultraviolet irradiation, inhibition on prostaglandin biosynthesis by ultraviolet irradiation, antibacterial action against acne bacteria, anti-inflammation action against atopic dermatitis, reduction of skin irritation, prevention of hair damage, prevention of alopecia and hair growth, thus being used for multifunctional compositions for skin external preparations.

Meanwhile, the composition for skin external application according to the present invention preferably contains 0.001 to 90.0% by weight of the Pleurotus ferulae fruiting body extract, the Pleurotus ferulae mycelium extract or the Pleurotus ferulae mycelium culture, based on the total weight of the composition. When the content of the Pleurotus ferulae fruiting body extract, the Pleurotus ferulae mycelium extract or the Pleurotus ferulae mycelium culture is lower than 0.001% by weight, skincare effects are insufficient and when the content thereof exceeds 90.0% by weight, economic efficiency is deteriorated due to low efficiency as compared to amounts of added materials.

Meanwhile, as used herein, the expression “contained as an active ingredient” means that the Pleurotus ferulae fruiting body extract, the Pleurotus ferulae mycelium extract or the Pleurotus ferulae mycelium culture is contained in the composition for skin external application of the present invention in an amount effective in imparting the effects of prevention and inhibition of skin diseases to the composition and means that the composition further contains various ingredients as secondary ingredients for drug delivery and stabilization, and is formulated in various forms.

Meanwhile, the composition for skin external application of the present invention is preferably a cosmetic composition. Examples of the formulation for the cosmetic composition include: cosmetic formulations for skin care including toner, gel, water-soluble liquid, cream, essence and oil-in-water (O/W) or water-in-oil (W/O) type emulsions; and cosmetic formulations for make-up including oil-in-water (O/W) or water-in-oil (W/O) type makeup base, foundation, skin cover, lipstick, lip gloss, face powder, two-way cake, eye-shadow, cheek color and eyebrow pencil.

Meanwhile, the cosmetic composition is most preferably utilized in applications of skin whitening, aging prevention, skin irritation reduction, skin moisturizing, wrinkle alleviation, atopy skin alleviation, acne alleviation, hair damage prevention or alopecia prevention.

Meanwhile, the composition for skin external application of the present invention is preferably a pharmaceutical composition. Examples of the formulation for the pharmaceutical composition include plasters, lotions, liniments, liquids and solutions, aerosols, extracts, ointments, fluidextracts, emulsions, suspensions, capsules, creams, soft or hard gelatin capsules, patches, and sustained release formulations.

Meanwhile, the pharmaceutical composition of the present invention may further comprise: pharmaceutically acceptable matrices; carriers; excipients; binders including starch, tragacanth rubber, gelatin, molasses, polyvinyl alcohol, polyvinyl ether, polyvinyl pyrrolidone, hydroxypropyl cellulose, methyl cellulose, ethyl cellulose, and carboxymethyl cellulose; disintegration agents including agar, starch, gelatin powder, sodium carboxymethyl cellulose, calcium carboxymethyl cellulose, microcrystalline cellulose calcium carbonate, sodium hydrogen carbonate and sodium alginate; lubricants including magnesium stearate, talc and hydrogenated vegetable oils; and coloring agents. Examples of the carriers and the excipients include lactose, glucose, sucrose, mannitol, potato starch, corn starch, calcium carbonate, calcium phosphate and cellulose and the like. The pharmaceutical composition may further comprise a stabilizer, a solubilizer, an adjuvant such as a dermal absorption accelerator, a flavoring agent, a preservative and the like.

Meanwhile, a standard dosage of the composition for skin external application administered to many patients may be changed according to individual characteristics of patients. Substantially, skilled clinical physicians determine an optimum treatment strategy in terms of an ideal dose and administration plans of the composition for skin external application for patients, for example, taking into consideration certain requirements and overall conditions of patients. The suitable dosage of the composition for skin external application may be determined as described in numerous reference documents.

In addition, the suitable dosage of the composition for skin external application may be generally determined in vivo or in an animal model. For example, the suitable dose may be determined by adding in vivo different concentrations of the composition for skin external application to target cells.

Meanwhile, the pharmaceutical composition is preferably used to prevent or treat inflammatory diseases, atopy or acne.

### EXAMPLE

The present invention will be described in more detail with reference to the following Examples. The scope of the present invention is not limited to the following examples and covers modifications of the technical spirit substantially equivalent thereto.

### Example 1

### Preparation of Pleurotus ferulae Fruiting Body Extract

A fruiting body of Pleurotus ferulae was lyophilized and ground to obtain a fruiting body powder, the fruiting body powder was extracted under reflux in an aqueous solution containing 70% (v/v) ethanol for 5 hours three times, macerated and filtered through a Whatman #5 filter paper. After the filtered extract was concentrated under reduced pressure at 50° C. or less and lyophilized, the resulting substance was dissolved in a mixed solvent of purified water, ethanol, butylene glycol and propylene glycol to prepare an extract containing 15% by weight of lyophilized Pleurotus ferulae (hereinafter, referred to as “Pleurotus ferulae fruiting body extract”).

### Example 2

### Preparation of Pleurotus ferulae mycelium Extract

A Pleurotus ferulae strain used for testing was directly isolated from a fresh fruiting body. A Pleurotus ferulae mycelium was slant-cultured in a test-tube containing a yeast-wort agar medium (containing 3 g of yeast extract, 3 g of wort, 10 g of glucose, 5 g of peptone, 20 g of agar and 1 L of distilled water), stored at 4° C. and sub-cultured once a month. The mycelium grown in the slant culture medium was aseptically homogenized, inoculated at a concentration of 5% (v/v) in a liquid medium obtained by adjusting a pH of a simple complex medium containing 3% glucose and 1.0% yeast extract to 5.5 and was cultured in a fermenter at a temperature of 27° C. at 150 rpm and at an air flow rate of 1.5 vvm for 10 days.

After culturing, the mycelium was collected by centrifugation and extracted by the method described in Example 1 to prepare a mycelium extract (hereinafter, referred to as “Pleurotus ferulae mycelium extract”).

### Example 3

### Preparation of Liquid Culture Obtained by Culturing Pleurotus ferulae Mycelium

A Pleurotus ferulae strain was directly isolated from a fresh fruiting body. Pleurotus ferulae mycelium was slant-cultured in a test-tube containing a yeast-wort agar medium (containing 3 g of yeast extract, 3 g of wort, 10 g of glucose, 5 g of peptone, 20 g of agar, and 1 L of distilled water), stored at 4° C. and sub-cultured once a month. The mycelium grown in the slant culture medium was aseptically homogenized, inoculated at a concentration of 5% (v/v) in a liquid medium obtained by adjusting a pH of a simple complex medium containing 3% glucose and a 1.0% yeast extract to 5.5 and was cultured in a fermenter at a temperature of 27° C. at 150 rpm and at air flow rate of 1.5 vvm for 10 days.

After culturing, the mycelium was collected by centrifugation and the culture residue was filtered through a paper filter (Whatman No. 4) to obtain a filtrate (hereinafter, referred to as “Pleurotus ferulae mycelium culture”).

### Test Example 1

### Measurement of Antioxidative Effects Using NBT Method

In this test example, antioxidative effects of the Pleurotus ferulae fruiting body extract obtained in Example 1, the Pleurotus ferulae mycelium extract obtained in Example 2 and the Pleurotus ferulae mycelium culture obtained in Example 3 were measured using an NBT method.

In accordance with the NBT method, active oxygen is produced by xanthine and xanthine oxidase, and absorbance at a wavelength of 560 nm of a blue substance produced upon reaction between the active oxygen and nitro blue tetrazolium (NBT) is measured. In the present test example, an active oxygen scavenge rate was measured in accordance with the following method. In addition, BHT, a well-known antioxidant, was used as a positive control group.

2.4 mL of 0.05M sodium carbonate (Na₂CO₃), 0.1 mL of a 3 mM xanthine solution, 0.1 mL of a 3 mM EDTA solution, 0.1 mL of a BSA solution and 0.1 mL of a 0.72 mM NBT solution were added to vials, 0.025, 0.05, 0.1 and 0.25% by weight of sample solutions were added respectively thereto, followed by allowing to stand at 25° C. for 10 minutes. After standing, 0.1 mL of a xanthine oxidase solution was added to the each solution which was then rapidly stirred and cultured at 25° C. for 20 minutes. 0.1 mL of a 6 mM copper chloride (CuCl₂) solution was added to the culture solution to stop the reaction. Absorbance (St) at 560 nm was measured. A blank test was carried out using distilled water instead of the sample solution. Absorbance (Bt) was measured in the same manner as above. Absorbance (Bo) of the blank of the sample solution was measured in the same manner as above using distilled water instead of the xanthine oxidase solution. In addition, active oxygen scavenge rate (%) was calculated by the following Equation 1.

Active oxygen scavenge rate (%)=[1−(St−So)/(Bt−Bo)]×100 Equation 1

St: absorbance at 560 nm of sample solution after enzyme reaction
Bt: absorbance at 560 nm of blank test solution after enzyme reaction
So: absorbance at 560 nm of enzyme-free sample solution before enzyme reaction
Bo: absorbance at 560 nm of enzyme-free blank test solution before enzyme reaction

TABLE 1

| | Active oxygen scavenge rate |
|---|---|
| Sample name | (antioxidative effect, %) |
| Pleurotus ferulae fruiting body | 95 |
| extract 0.25 wt % | |
| Pleurotus ferulae fruiting body | 92 |
| extract 0.1 wt % | |
| Pleurotus ferulae fruiting body | 78 |
| extract 0.05 wt % | |
| Pleurotus ferulae fruiting body | 41 |
| extract 0.025 wt % | |
| BHT 0.1 wt % | 89 |

TABLE 2

| | Active oxygen scavenge rate |
|---|---|
| Sample name | (antioxidative effect, %) |
| Pleurotus ferulae mycelium extract | 94 |
| 0.25 wt % | |
| Pleurotus ferulae mycelium extract | 90 |
| 0.1 wt % | |
| Pleurotus ferulae mycelium extract | 76 |
| 0.05 wt % | |
| Pleurotus ferulae mycelium extract | 42 |
| 0.025 wt % | |
| BHT 0.1 wt % | 89 |

TABLE 3

| | Active oxygen scavenge rate |
|---|---|
| Sample name | (antioxidative effect, %) |
| Pleurotus ferulae mycelium culture | 94 |
| 0.25 wt % | |
| Pleurotus ferulae mycelium culture | 91 |
| 0.1 wt % | |
| Pleurotus ferulae mycelium culture | 75 |
| 0.05 wt % | |
| Pleurotus ferulae mycelium culture | 39 |
| 0.025 wt % | |
| BHT 0.1 wt % | 89 |

As can be seen from results (Tables 1 to 3) of measurement of antioxidative effects using the NBT method, all of the Pleurotus ferulae fruiting body extract, the Pleurotus ferulae mycelium extract and the Pleurotus ferulae mycelium culture exhibit superior antioxidative effects to BHT at the same concentration.

From the results, it can be seen that the Pleurotus ferulae fruiting body extract, the Pleurotus ferulae mycelium extract and the Pleurotus ferulae mycelium culture exhibit superior antioxidative effects. This demonstrates that the composition for skin external application according to the present invention exhibits superior antioxidative effects.

### Test Example 2

### Measurement of Antioxidative Effects Using DPPH Method

In the present test example, antioxidative effects of the Pleurotus ferulae fruiting body extract obtained in Example 1, the Pleurotus ferulae mycelium extract obtained in Example 2 and the Pleurotus ferulae mycelium culture obtained in Example 3 were measured using a DPPH method.

The DPPH method measures free radical scavenge activity based on reduction power using a free radical, called “DPPH (2,2-di(4-tert-octylphenyl)-1-picrylhydrazyl)”. In the present test example, decrease in absorbance caused by reduction of DPPH by a test substance was compared with an absorbance of a blank test solution, and free radical scavenge rate at a wavelength of 560 nm was measured. Measurement was performed as follows. In addition, BHT, a well-known antioxidant, was used as a positive control group.

0.025, 0.05, 0.1 and 0.25% by weight of different concentrations of the Pleurotus ferulae fruiting body extracts, the Pleurotus ferulae mycelium extracts and the Pleurotus ferulae mycelium cultures were prepared and pipetted into on 96-well plates, and DPPH prepared from a 100 μM methanol solution was added thereto such that the total volume of the solution reached 200 μl. The solution was allowed to stand at 37° C. for 30 minutes, absorbance (St) at 560 nm of the solution was measured and free radical scavenge activity (%) was calculated using the following Equation 2.

Free radical scavenge activity (%)={100−(B/A)}×100 Equation 2

A: absorbance of control group well not treated with Pleurotus ferulae fruiting body extract, Pleurotus ferulae mycelium extract or Pleurotus ferulae mycelium culture
B: absorbance of experimental group well treated with Pleurotus ferulae fruiting body extract, Pleurotus ferulae mycelium extract or Pleurotus ferulae mycelium culture

TABLE 4

| | Free radical scavenge rate |
|---|---|
| Sample name | (antioxidative effect, %) |
| Pleurotus ferulae fruiting body | 95 |
| extract 0.25 wt % | |
| Pleurotus ferulae fruiting body | 93 |
| extract 0.1 wt % | |
| Pleurotus ferulae fruiting body | 88 |
| extract 0.05 wt % | |
| Pleurotus ferulae fruiting body | 64 |
| extract 0.025 wt % | |
| BHT 0.1 wt % | 85 |

TABLE 5

| | Free radical scavenge rate |
|---|---|
| Sample name | (antioxidative effect, %) |
| Pleurotus ferulae mycelium extract | 94 |
| 0.25 wt % | |
| Pleurotus ferulae mycelium extract | 91 |
| 0.1 wt % | |
| Pleurotus ferulae mycelium extract | 86 |
| 0.05 wt % | |
| Pleurotus ferulae mycelium extract | 61 |
| 0.025 wt % | |
| BHT 0.1 wt % | 85 |

TABLE 6

| | Free radical scavenge rate |
|---|---|
| Sample name | (antioxidative effect, %) |
| Pleurotus ferulae mycelium culture | 91 |
| 0.25 wt % | |
| Pleurotus ferulae mycelium culture | 89 |
| 0.1 wt % | |
| Pleurotus ferulae mycelium culture | 82 |
| 0.05 wt % | |
| Pleurotus ferulae mycelium culture | 59 |
| 0.025 wt % | |
| BHT 0.1 wt % | 85 |

As can be seen from results (Tables 4 to 6) of measurement of antioxidative effects using the DPPH method, all of the Pleurotus ferulae fruiting body extract, the Pleurotus ferulae mycelium extract and the Pleurotus ferulae mycelium culture exhibit superior antioxidative effects to BHT at the same concentration.

From the results, it can be seen that the Pleurotus ferulae fruiting body extract, the Pleurotus ferulae mycelium extract and the Pleurotus ferulae mycelium culture exhibit superior antioxidative effects. This demonstrates that the composition for skin external application according to the present invention exhibits superior antioxidative effects.

### Test Example 3

### Measurement of Effect of Scavenging Active Oxygen in Cells

In the present test example, the effect of the Pleurotus ferulae fruiting body extract obtained in Example 1, the Pleurotus ferulae mycelium extract obtained in Example 2 and the Pleurotus ferulae mycelium culture obtained in Example 3 on scavenging of active oxygen produced by ultraviolet radiation was measured.

A cell line used for the present test example was a Human keratinocytes HaCaT cell line obtained from Dr. Fusenig in the German Cancer Research Center. The cell line was seeded at a density of 2.0×10⁴ cells per well into 96-well black plates for fluorescent measurement and cultured in DMEM (Dulbecco's Modified Eagle's Medium, FBS 10%, Gibco, USA) media containing penicillin and streptomycin at 37° C. in an atmosphere containing 5% CO₂ for one day. Then, the culture media were respectively treated with the Pleurotus ferulae fruiting body extract, the Pleurotus ferulae mycelium extract and the Pleurotus ferulae mycelium culture at different concentrations of 0.005, 0.01 and 0.02% by weight.

After test samples were cultured for 24 hours, the test samples were washed with HCSS (HEPES-buffered control salt solution) to remove the remaining media, 100 μl of a solution of 20 μM DCFH-DA (2′,7′-dichlorodihydro-fluorescein diacetate, Molecular Probes, USA) in HCSS was added to the cells, and the cells were cultured at 37° C. in an atmosphere containing 5% CO₂ for 20 minutes and washed with HCSS. Then, after 100 μl of different concentrates of HCSS was added to the test samples, fluorescence of DCF (dichlorofluorescein) initially oxidized with active oxygen was measured using a fluorescence plate reader (Ex: 485 nm, Em: 530 nm). Then, the samples were irradiated with UVB (20 mJ/cm²), treated with 0.005, 0.01 and 0.02% by weight of the Pleurotus ferulae fruiting body extract, the Pleurotus ferulae mycelium extract and the Pleurotus ferulae mycelium culture and then measured for fluorescence using the fluorescence plate reader (Ex: 485 nm, Em: 530 nm). At this time, epigallocatechin gallate (EGCG) known to have potent antioxidative effects was used as a positive control group.

TABLE 7

| | Effect of scavenging active oxygen |
|---|---|
| Sample name | (%) |
| Pleurotus ferulae fruiting body | 56 |
| extract 0.02 wt % | |
| Pleurotus ferulae fruiting body | 49 |
| extract 0.01 wt % | |
| Pleurotus ferulae fruiting body | 23 |
| extract 0.005 wt % | |
| EGCG 0.01 wt % | 45 |

TABLE 8

| | Effect of scavenging |
|---|---|
| Sample name | active oxygen (%) |
| Pleurotus ferulae mycelium | 55 |
| extract 0.02 wt % | |
| Pleurotus ferulae mycelium | 49 |
| extract 0.01 wt % | |
| Pleurotus ferulae mycelium | 22 |
| extract 0.005 wt % | |
| EGCG 0.01 wt % | 45 |

TABLE 9

| | Effect of scavenging |
|---|---|
| Sample name | active oxygen (%) |
| Pleurotus ferulae mycelium | 55 |
| culture 0.02 wt % | |
| Pleurotus ferulae mycelium | 47 |
| culture 0.01 wt % | |
| Pleurotus ferulae mycelium | 19 |
| culture 0.005 wt % | |
| EGCG 0.01 wt % | 45 |

As can be seen from results (Tables 7 to 9) of measurement of the effect of scavenging active oxygen, all of the Pleurotus ferulae fruiting body extract, the Pleurotus ferulae mycelium extract and the Pleurotus ferulae mycelium culture exhibit higher active oxygen scavenge rates than EGCG at the same concentration.

From the results, it can be seen that the Pleurotus ferulae fruiting body extract, the Pleurotus ferulae mycelium extract and the Pleurotus ferulae mycelium culture are potently effective in scavenging active oxygen. This demonstrates that the composition for skin external application according to the present invention is potently effective in scavenging active oxygen.

### Test Example 4

### Measurement of Inhibition on Matrix Metalloproteinase (MMP-1) Activity (In Vitro)

In the present test example, the inhibition rate of matrix metalloproteinase (MMP-1) activity (in vitro) of the Pleurotus ferulae fruiting body extract obtained in Example 1, the Pleurotus ferulae mycelium extract obtained in Example 2 and the Pleurotus ferulae mycelium culture obtained in Example 3 was measured using fluorescence analysis.

A substrate and enzyme used for the present test were fluorescein-labeled DQ-Collagen and collagenase available from Molecular probes (Eugene, Oreg., USA), respectively, and a buffer {0.5M Tris-HCl, 1.5 M NaCl, 50 mM CaCl₂, 2 mM sodium azide, pH 7.6} solution was diluted 10-fold before use. A solution (0.25 mg/mL) of 20 μl of DQ collagen in a buffer solution and 40 μl of the Pleurotus ferulae fruiting body extract, the Pleurotus ferulae mycelium extract and the Pleurotus ferulae mycelium culture (at concentrations of 0.005, 0.01 and 0.02% by weight) were added to 100 μl of the buffer solution, and 40 μl of a collagenase dilution (0.5 units) was added thereto. After the solution was stored in a dark room at room temperature for 20 minutes, fluorescence of the solution was measured at an absorption wavelength of 495 nm and an emission wavelength of 515 nm using a spectrophotofluorometer (Perkin Elmer, UK). In addition, a green tea extract was used as a positive control group. Fluorescence of the control group was measured after adding an equivalent amount of the buffer solution, instead of the enzyme solution. In addition, fluorescence of the sample was measured and was used for calculation of enzyme activity.

TABLE 10

| | Inhibition of MMP-1 |
|---|---|
| Sample name | activity (%) |
| Control group | 0 |
| Pleurotus ferulae fruiting | 73 |
| body extract 0.02 wt % | |
| Pleurotus ferulae fruiting | 59 |
| body extract 0.01 wt % | |
| Pleurotus ferulae fruiting | 40 |
| body extract 0.005 wt % | |
| Green tea extract 0.2 wt % | 68 |

TABLE 11

| | Inhibition of MMP-1 |
|---|---|
| Sample name | activity (%) |
| Control group | 0 |
| Pleurotus ferulae mycelium | 74 |
| extract 0.02 wt % | |
| Pleurotus ferulae mycelium | 56 |
| extract 0.01 wt % | |
| Pleurotus ferulae mycelium | 41 |
| extract 0.005 wt % | |
| Green tea extract 0.2 wt % | 68 |

TABLE 12

| | Inhibition of MMP-1 |
|---|---|
| Sample name | activity (%) |
| Control group | 0 |
| Pleurotus ferulae mycelium | 71 |
| culture 0.02 wt % | |
| Pleurotus ferulae mycelium | 58 |
| culture 0.01 wt % | |
| Pleurotus ferulae mycelium | 37 |
| culture 0.005 wt % | |
| Green tea extract 0.2 wt % | 68 |

As can be seen from results (Tables 10 to 12) of measurement of inhibition on matrix metalloproteinase (MMP-1) activity in vitro, all of the Pleurotus ferulae fruiting body extract, the Pleurotus ferulae mycelium extract and the Pleurotus ferulae mycelium culture effectively inhibit matrix metalloproteinase (MMP-1) activity at a low concentration, as compared to the green tea extract.

From the results, it can be seen that the Pleurotus ferulae fruiting body extract, the Pleurotus ferulae mycelium extract and the Pleurotus ferulae mycelium culture are potently effective in inhibiting matrix metalloproteinase (MMP-1) activity. This demonstrates that the composition for skin external application according to the present invention effectively inhibits matrix metalloproteinase (MMP-1) activity and thereby prevents deterioration in elasticity of the skin and formation of wrinkles.

### Test Example 5

### Measurement of Repression of Matrix Metalloproteinase (MMP-1) after Ultraviolet Irradiation

In the present test example, the effects of the Pleurotus ferulae fruiting body extract obtained in Example 1, the Pleurotus ferulae mycelium extract obtained in Example 2 and the Pleurotus ferulae mycelium culture obtained in Example 3 on repression of matrix metalloproteinase (MMP-1) after ultraviolet irradiation were measured.

After UV irradiation and addition of samples (Pleurotus ferulae fruiting body extract, Pleurotus ferulae mycelium extract or Pleurotus ferulae mycelium culture, retinol), enzyme-linked immunosorbent assay (ELISA) was carried out to measure concentration of matrix metalloproteinase (MMP-1).

Human dermal fibroblasts were irradiated with UVA at an energy of 6.3 J/cm² in a UV chamber, and conditions of the dose of ultraviolet radiation and culture time which maximize expression of matrix metalloproteinase (MMP-1) in fibroblasts were established through preliminary testing. A negative control group was wrapped with a foil and exposed to UVA environment for the same time. Here, UVA dose was measured using a UV radiometer. The cells during irradiation of UVA were cultured in the medium previously used. After UVA irradiation, the cells were cultured in a fresh medium containing samples for 24 hours and the medium were coated on 96-well plates. The cells were treated with primary antibody {MMP-1 (Ab-5) monoclonal antibody} and reaction was conducted at 37° C. for 60 minutes. The cells were reacted with anti-mouse IgG (whole mouse, alkaline phosphatase conjugated) as a secondary antibody for about 60 minutes, and an alkaline phosphatase substrate solution (1 mg/mL ρ-nitrophenyl phosphate in diethanolamine buffer solution) at room temperature for 30 minutes, and absorbance at 405 nm was measured using a microplate reader. A control group was not treated with the sample and retinol was used as a positive control group.

TABLE 13

| | Repression of |
|---|---|
| Sample name | MMP-1 (%) |
| Control group | 0 |
| Pleurotus ferulae fruiting | 66 |
| body extract 0.02 wt % | |
| Pleurotus ferulae fruiting | 31 |
| body extract 0.01 wt % | |
| Pleurotus ferulae fruiting | 19 |
| body extract 0.005 wt % | |
| retinol 0.02% by weight | 36 |

TABLE 14

| | Repression of |
|---|---|
| Sample name | MMP-1 (%) |
| Control group | 0 |
| Pleurotus ferulae mycelium | 65 |
| extract 0.02 wt % | |
| Pleurotus ferulae mycelium | 32 |
| extract 0.01 wt % | |
| Pleurotus ferulae mycelium | 22 |
| extract 0.005 wt % | |
| Retinol 0.02% by weight | 36 |

TABLE 15

| | Repression of |
|---|---|
| Sample name | MMP-1 (%) |
| Control group | 0 |
| Pleurotus ferulae mycelium | 65 |
| culture 0.02 wt % | |
| Pleurotus ferulae mycelium | 33 |
| culture 0.01 wt % | |
| Pleurotus ferulae mycelium | 21 |
| culture 0.005 wt % | |
| Retinol 0.02% by weight | 36 |

As can be seen from the results (Tables 13 to 15) of measurement of repression of matrix metalloproteinase (MMP-1) after ultraviolet irradiation, all of the Pleurotus ferulae fruiting body extract, the Pleurotus ferulae mycelium extract and the Pleurotus ferulae mycelium culture exhibit good repression activity, as compared to retinol, the positive control group.

From the results, it can be seen that the Pleurotus ferulae fruiting body extract, the Pleurotus ferulae mycelium extract and the Pleurotus ferulae mycelium culture are potently effective in repressing matrix metalloproteinase (MMP-1). This demonstrates that the composition for skin external application according to the present invention effectively represses matrix metalloproteinase (MMP-1) and thereby prevents deterioration in elasticity of the skin and formation of wrinkles.

### Test Example 6

### Measurement of Effect of Promoting Type 1 Procollagen Biosynthesis

In the present test example, the effect of the Pleurotus ferulae fruiting body extract obtained in Example 1, the Pleurotus ferulae mycelium extract obtained in Example 2 and the Pleurotus ferulae mycelium culture obtained in Example 3 on promotion of biosynthesis of type 1 procollagen, skin substrate ingredient, was measured.

Human dermal fibroblasts isolated from infant epidermal tissues were obtained from Modern Tissue Technology (MTT, Korea) and cultured at 1×10⁴ cell/cm₂ in a DMEM/F-12 (3:1) medium containing 10% fetal bovine serum (FBS). The human dermal fibroblasts grown to about a confluence of 80% were sub-cultured at a split ratio of 1:3 four times and were then used for the testing.

For the test to measure the amount of procollagen, fibroblasts were cultured to a confluence of 90% or more on a 48-well plate, the Pleurotus ferulae fruiting body extract, the Pleurotus ferulae mycelium extract and the Pleurotus ferulae mycelium culture were added at different concentrations of 0.005, 0.01 and 0.02% by weight to the fibroblasts, and after 24 hours, the amount of procollagen released in the medium was measured using a procollagen type-1C-peptide EIA kit (MK101, Takara, Japan). In addition, TGF-β was used as a positive control group.

TABLE 16

| | Promotion of procollagen |
|---|---|
| Sample name | biosynthesis (%) |
| Pleurotus ferulae fruiting | 143 |
| body extract 0.02 wt % | |
| Pleurotus ferulae fruiting | 134 |
| body extract 0.01 wt % | |
| Pleurotus ferulae fruiting | 126 |
| body extract 0.005 wt % | |
| TGF-β 0.001 wt % | 125 |

TABLE 17

| | Promotion of procollagen |
|---|---|
| Sample name | biosynthesis (%) |
| Pleurotus ferulae mycelium | 145 |
| extract 0.02 wt % | |
| Pleurotus ferulae mycelium | 136 |
| extract 0.01 wt % | |
| Pleurotus ferulae mycelium | 128 |
| extract 0.005 wt % | |
| TGF-β 0.001 wt % | 125 |

TABLE 18

| | Promotion of procollagen |
|---|---|
| Sample name | biosynthesis (%) |
| Pleurotus ferulae mycelium | 148 |
| culture 0.02 wt % | |
| Pleurotus ferulae mycelium | 137 |
| culture 0.01 wt % | |
| Pleurotus ferulae mycelium | 129 |
| culture 0.005 wt % | |
| TGF-β 0.001 wt % | 125 |

As can be seen from results (Tables 16 to 18) of measurement of promotion of procollagen biosynthesis, all of the Pleurotus ferulae fruiting body extract, the Pleurotus ferulae mycelium extract and the Pleurotus ferulae mycelium culture exhibit effects similar to TGF-β which is a signal transduction substance (messenger).

From the results, it can be seen that the Pleurotus ferulae fruiting body extract, the Pleurotus ferulae mycelium extract and the Pleurotus ferulae mycelium culture are potently effective in promoting procollagen biosynthesis. This demonstrates that the composition for skin external application according to the present invention exhibits potent anti-aging effects upon the skin.

### Test Example 7

### Measurement of Effect of Inhibiting Elastase Activity

In the present test example, the effect of the Pleurotus ferulae fruiting body extract obtained in Example 1, the Pleurotus ferulae mycelium extract obtained in Example 2 and the Pleurotus ferulae mycelium culture obtained in Example 3 on inhibition of elastase activity was measured.

Human fibroblasts were cultured to about 80% confluence in a flask for culture. Then, the fibroblasts were respectively treated with the Pleurotus ferulae fruiting body extract, the Pleurotus ferulae mycelium extract and the Pleurotus ferulae mycelium culture for one day, and the cell cultures were harvested and measured for elastin production using a commercially available elastin measurement apparatus [Bieth J: Biochem med., 11, 350-357 (1974), Schwartz D E: J. Invest Dermatol., 86, 63-68 (1986)]. That is, activity of elastase was measured using absorbance based on change in color caused by decomposition of NA of Suc-(Ala) 3 NA which is a substrate of elastase. At this time, a group not treated with the Pleurotus ferulae fruiting body extract, the Pleurotus ferulae mycelium extract and the Pleurotus ferulae mycelium culture was used as a control group.

TABLE 19

| | Inhibition of elastase |
|---|---|
| Sample name | activity (%) |
| Control group | 0 |
| Pleurotus ferulae fruiting | 30 |
| body extract 0.02 wt % | |
| Pleurotus ferulae fruiting | 19 |
| body extract 0.01 wt % | |
| Pleurotus ferulae fruiting | 10 |
| body extract 0.005 wt % | |

TABLE 20

| | Inhibition of elastase |
|---|---|
| Sample name | activity (%) |
| Control group | 0 |
| Pleurotus ferulae mycelium | 29 |
| extract 0.02 wt % | |
| Pleurotus ferulae mycelium | 18 |
| extract 0.01 wt % | |
| Pleurotus ferulae mycelium | 8 |
| extract 0.005 wt % | |

TABLE 21

| | Inhibition of elastase |
|---|---|
| Sample name | activity (%) |
| Control group | 0 |
| Pleurotus ferulae mycelium | 29 |
| culture 0.02 wt % | |
| Pleurotus ferulae mycelium | 16 |
| culture 0.01 wt % | |
| Pleurotus ferulae mycelium | 9 |
| culture 0.005 wt % | |

As can be seen from results (Tables 19 to 21) of measurement of inhibition of elastase activity, all of the Pleurotus ferulae fruiting body extract, the Pleurotus ferulae mycelium extract and the Pleurotus ferulae mycelium culture exhibit a concentration-dependent increase in inhibition of elastase activity.

From the results, it can be seen that the Pleurotus ferulae fruiting body extract, the Pleurotus ferulae mycelium extract and the Pleurotus ferulae mycelium culture are potently effective in inhibiting elastase activity. This demonstrates that the composition for skin external application according to the present invention exhibits potent anti-aging and elasticity enhancement effects.

### Test Example 8

### Measurement of Effect of Inhibiting Tyrosinase Activity

In the present test example, the effects of the Pleurotus ferulae fruiting body extract obtained in Example 1, the Pleurotus ferulae mycelium extract obtained in Example 2 and the Pleurotus ferulae mycelium culture obtained in Example 3 on inhibition of tyrosinase activity were measured.

Tyrosinase is an enzyme which facilitates oxidation of a substance called “tyrosine” in vivo and aids in production of melanin. Tyrosinase was isolated from mushrooms and purified, and was obtained from Sigma Aldrich Corp. A 0.1 mg/mL solution of L-tyrosine as a substrate in a 0.05 M sodium phosphate buffer solution (pH 6.8) was prepared. The Pleurotus ferulae fruiting body extract, the Pleurotus ferulae mycelium extract and the Pleurotus ferulae mycelium culture were dissolved at different concentrations of 0.005, 0.01 and 0.02% by weight in 0.05M sodium phosphate buffer solutions (pH 6.8). 0.5 mL of the L-tyrosine solution was added to a test tube, 0.5 mL of the Pleurotus ferulae fruiting body extract, Pleurotus ferulae mycelium extract and Pleurotus ferulae mycelium culture sample solutions were added to the L-tyrosine solution, and the resulting solution was incubated in a 37° C. incubator for 10 minutes. Then, the solution was reacted with 0.5 mL of 200 unit/mL tyrosinase at 37° C. for 10 minutes. The test tube containing the reaction solution was quenched in ice to stop the reaction and transmissivity at a wavelength of 475 nm was measured using a spectrophotometer. A group treated with 0.5 mL of a buffer solution, instead of the Pleurotus ferulae fruiting body extract, the Pleurotus ferulae mycelium extract or the Pleurotus ferulae mycelium culture was used as a control group, and arbutin, a well-known whitening agent, was used as a positive control group. In addition, inhibition of tyrosinase activity by the respective samples were calculated in accordance with the following Equation 3.

Inhibition of tyrosinase activity (%)=100−[(absorbance of reference group/absorbance of control group)]×100 Equation 3

TABLE 22

| | Inhibition of tyrosinase |
|---|---|
| Sample name | activity (%) |
| Control group | 0 |
| Pleurotus ferulae fruiting | 56 |
| body extract 0.02 wt % | |
| Pleurotus ferulae fruiting | 44 |
| body extract 0.01 wt % | |
| Pleurotus ferulae fruiting | 31 |
| body extract 0.005 wt % | |
| Arbutin 0.2 wt % | 54 |

TABLE 23

| | Inhibition of tyrosinase |
|---|---|
| Sample name | activity (%) |
| Control group | 0 |
| Pleurotus ferulae mycelium | 55 |
| extract 0.02 wt % | |
| Pleurotus ferulae mycelium | 43 |
| extract 0.01 wt % | |
| Pleurotus ferulae mycelium | 33 |
| extract 0.005 wt % | |
| Arbutin 0.2 wt % | 54 |

TABLE 24

| | Inhibition of tyrosinase |
|---|---|
| Sample name | activity (%) |
| Control group | 0 |
| Pleurotus ferulae mycelium | 51 |
| culture 0.02 wt % | |
| Pleurotus ferulae mycelium | 41 |
| culture 0.01 wt % | |
| Pleurotus ferulae mycelium | 35 |
| culture 0.005 wt % | |
| Arbutin 0.2 wt % | 54 |

As can be seen from results (Tables 22 to 24) of measurement of inhibition on tyrosinase activity, all of the Pleurotus ferulae fruiting body extract, the Pleurotus ferulae mycelium extract and the Pleurotus ferulae mycelium culture exhibit inhibition against tyrosinase activity similar to albutin.

From the results, it can be seen that the Pleurotus ferulae fruiting body extract, the Pleurotus ferulae mycelium extract and the Pleurotus ferulae mycelium culture are potently effective in inhibiting tyrosinase activity. This demonstrates that the composition for skin external application according to the present invention exhibits potent whitening effect.

### Test Example 9

### Measurement of Effect of Inhibiting Melanin Synthesis Using B16F1 Melanin Cells

In the present test example, the effects of the Pleurotus ferulae fruiting body extract obtained in Example 1, the Pleurotus ferulae mycelium extract obtained in Example 2 and Pleurotus ferulae mycelium culture obtained in Example 3 on inhibition of melanin synthesis were measured.

B16F1 melanocytes used for the present test example were cell lines derived from mice, which secrete a black pigment called “melanin”. The effect on inhibition of melanin synthesis of B16F1 melanin cells was measured as follows. B16F1 melanin cells were seeded at a density of 2×10⁶ cells per well on a 6-well plate, treated with 0.005, 0.01 and 0.02% by weight of the respective samples and cultured for 72 hours. After culturing for 72 hours, the cells were removed from trypsin-EDTA, counted and collected by centrifugation. Melanin in cells was assayed using a slightly modified version of Lotan's method (R Lotan and D Lotan, Cancer Res, 40: 3345-3350, 1980). Cell pellets were washed with PBS once and homogenized, 1 mL of a buffer solution (50 mM sodium phosphate, pH 6.8, 1% Triton X-100, 2 mM PMSF) was added to the cells, and the cells were crushed by vortex for 5 minutes. Melanin extracted by addition of 1N NaOH (10% DMSO) to a cell residue obtained by centrifugation (3,000 rpm, 10 minutes) was dissolved, absorbance of melanin at 405 nm was measured using a microplate reader, melanin was assayed, and inhibition of melanin production (%) of the sample was measured. In addition, a group not treated with the Pleurotus ferulae fruiting body extract, the Pleurotus ferulae mycelium extract or the Pleurotus ferulae mycelium culture was used as a control group, arbutin well-known as a whitening agent was used as a positive control group, and inhibition of melanin synthesis (%) by B16F1 melanin cells was calculated in accordance with Equation 4.

Inhibition of melanin synthesis (%)=[(A−B)/A]×100 Equation 4

A: amount of melanin in well containing no sample
B: amount of melanin in well containing sample

TABLE 25

| | Inhibition of melanin |
|---|---|
| Sample name | synthesis (%) |
| Control group | 0 |
| Pleurotus ferulae fruiting | 68 |
| body extract 0.02 wt % | |
| Pleurotus ferulae fruiting | 51 |
| body extract 0.01 wt % | |
| Pleurotus ferulae fruiting | 42 |
| body extract 0.005 wt % | |
| Arbutin 0.2 wt % | 55 |

TABLE 26

| | Inhibition of melanin |
|---|---|
| Sample name | synthesis (%) |
| Control group | 0 |
| Pleurotus ferulae mycelium | 67 |
| extract 0.02 wt % | |
| Pleurotus ferulae mycelium | 51 |
| extract 0.01 wt % | |
| Pleurotus ferulae mycelium | 40 |
| extract 0.005 wt % | |
| Arbutin 0.2 wt % | 55 |

TABLE 27

| | Inhibition of melanin |
|---|---|
| Sample name | synthesis (%) |
| Control group | 0 |
| Pleurotus ferulae mycelium | 66 |
| culture 0.02 wt % | |
| Pleurotus ferulae mycelium | 52 |
| culture 0.01 wt % | |
| Pleurotus ferulae mycelium | 39 |
| culture 0.005 wt % | |
| Arbutin 0.2 wt % | 55 |

As can be seen from results (Tables 25 to 27) of measurement of inhibition activity on melanin synthesis using B16F1 melanin cells, all of the Pleurotus ferulae fruiting body extract, the Pleurotus ferulae mycelium extract and the Pleurotus ferulae mycelium culture exhibit inhibitory activity on melanin synthesis similar to albutin.

From the results, it can be seen that the Pleurotus ferulae fruiting body extract, the Pleurotus ferulae mycelium extract and the Pleurotus ferulae mycelium culture are potently effective in inhibiting melanin synthesis. This demonstrates that the composition for skin external application according to the present invention exhibits potent skin whitening effects.

### Test Example 10

### Measurement of Effect of Inhibiting Inflammation-Inducing Enzyme (Hyaluronidase) Activity

In the present test example, the inhibitory effects against hyaluronidase activity of the Pleurotus ferulae fruiting body extract obtained in Example 1, the Pleurotus ferulae mycelium extract obtained in Example 2 and the Pleurotus ferulae mycelium culture obtained in Example 3 was measured.

Hyaluronidase is an enzyme which hydrolyzes hyaluronic acid and induces inflammation. In the present test example, anti-inflammatory effects were evaluated by measuring anti-inflammatory effects through inhibition of hyaluronidase activity (Kakegawa Y: Japanese J. of Inflammation, 4, 437-438, 1984). In addition, comfrey, Shiso, fat-soluble licorice extracts were used as positive control groups.

100 μl of each sample was reacted with 50 μl of a hyaluronidase solution (type IV-S, Sigma, 400 U/mL) at 37° C. for 20 minutes, and the resulting solution was reacted with 100 μl of an enzyme-activating solution (compound 48/80 CaCl₂.2H₂O, Sigma, 0.1 mg/mL) at 37° C. for 20 minutes again. The resulting solution was reacted with 250 μl of a hyaluronic acid solution (0.4 mg/mL) at 37° C. for 40 minutes, and 100 μl of 0.4N NaOH was added to the reaction solution to stop the reaction. The resulting solution was reacted with 100 μl of a potassium borate solution at 95° C. for 3 minutes, followed by cooling. The resulting solution was reacted with 3 mL of a ρ-dimethylaminobenzaldehyde solution at 37° C. for 20 minutes, followed by color development.

Absorbance at 585 nm was measured to evaluate inhibition of hyaluronidase activity. Inhibition of hyaluronidase activity (%) was calculated in accordance with the following Equation 5, and IC50 is a concentration of a substance inhibiting hyaluronidase enzyme activity by 50%.

Inhibition of hyaluronidase activity (%)=[(A−B)/A]×100 Equation 5

A: enzyme activity of well containing no sample
B: enzyme activity of well containing sample

TABLE 28

| | Inhibition of hyaluronidase |
|---|---|
| Sample name | activity (IC_{50,} %) |
| Comfrey extract | 0.25 |
| Shiso extract | 0.50 |
| Fat-soluble licorice extract | 0.20 |
| Pleurotus ferulae fruiting | 0.25 |
| body extract | |

TABLE 29

| | Inhibition of hyaluronidase |
|---|---|
| Sample name | activity (IC_{50,} %) |
| Comfrey extract | 0.25 |
| Shiso extract | 0.50 |
| Fat-soluble licorice extract | 0.20 |
| Pleurotus ferulae | 0.25 |
| mycelium extract | |

TABLE 30

| | Inhibition of hyaluronidase |
|---|---|
| Sample name | activity (IC_{50,} %) |
| Comfrey extract | 0.25 |
| Shiso extract | 0.50 |
| Fat-soluble licorice extract | 0.20 |
| Pleurotus ferulae | 0.25 |
| mycelium culture | |

As can be seen from results (Tables 28 to 30) of measurement of inhibition of hyaluronidase activity, all IC₅₀ values of the Pleurotus ferulae fruiting body extract, the Pleurotus ferulae mycelium extract and the Pleurotus ferulae mycelium culture exhibit similar or superior effects to the comfrey, Shiso and fat-soluble licorice extracts well known as anti-inflammatory drugs.

From the results, it can be seen that the Pleurotus ferulae fruiting body extract, the Pleurotus ferulae mycelium extract and the Pleurotus ferulae mycelium culture exhibit superior anti-inflammatory effects. This demonstrates that the composition for skin external application according to the present invention exhibits potent anti-inflammatory effects.

### Test Example 11

### Measurement of Effect of Reducing Cytotoxicity Induced by Ultraviolet Irradiation

In the present test example, the effect of the Pleurotus ferulae fruiting body extract obtained in Example 1, the Pleurotus ferulae mycelium extract obtained in Example 2 and the Pleurotus ferulae mycelium culture obtained in Example 3 on reduction of cytotoxicity induced by ultraviolet irradiation was measured.

Fibroblasts were seeded at a density of 1×10⁵ cells on a 24-well test plate for 24 hours. Each well was washed with PBS once and 500 μl of PBS was added thereto. The fibroblasts were irradiated with 10 mJ/cm² of ultraviolet light using a ultraviolet B (UVB) lamp (Model: F15T8, UVB 15W, Sankyo Denki Co., Ltd., Japan), PBS was removed and 1 mL of a cell culture medium (DMEM supplemented with 10% FBS) was added to the fibroblasts. The fibroblasts were treated with 0.005, 0.01 and 0.02% by weight of the Pleurotus ferulae fruiting body extract, the Pleurotus ferulae mycelium extract and the Pleurotus ferulae mycelium culture and cultured for 24 hours. After 24 hours, the medium was removed, 500 μl of a cell culture medium and 60 μl of a MTT solution (2.5 mg/mL) were added to each well and the cells were cultured in a CO₂ incubator at 37° C. for 2 hours. After culturing, the medium was removed and 500 μl of isopropanol-HCl (0.04N) was added to the residue. The cells were shaken for 5 minutes to induce cytolysis, 100 μl of a supernatant was added to a 96-well test plate, and absorbance was measured at 565 nm using a microplate reader. Cell survival rate (%) was calculated in accordance with the following Equation 6 and reduction of cytotoxicity induced by ultraviolet radiation was calculated in accordance with the following Equation 7.

Cell survival rate (%)=[(St−Bo)/(Bt−Bo)]×100 Equation 6

Bo: 565 nm at absorbance of well containing only cell culture medium and undergoing color reaction
Bt: 565 nm at absorbance of well not treated with sample and undergoing color reaction
St: 565 nm at absorbance of well treated with sample and undergoing color reaction

Reduction of cytotoxicity induced by ultraviolet radiation (%)=[1−(St−Bo)/(Bt−Bo)]×100 Equation 7

Bo: cell survival rate of well not irradiated with ultraviolet light and not treated with sample
Bt: cell survival rate of well irradiated with ultraviolet light and not treated with sample
St: cell survival rate of well irradiated with ultraviolet light and treated with sample

TABLE 31

| | Concentration of | Cytotoxicity |
|---|---|---|
| Sample name | treated sample (wt %) | reduction (%) |
| Pleurotus ferulae | 0.02 | 49 |
| fruiting body extract | 0.01 | 32 |
| | 0.005 | 18 |

TABLE 32

| | Concentration of | Cytotoxicity |
|---|---|---|
| Sample name | treated sample (wt %) | reduction (%) |
| Pleurotus ferulae | 0.02 | 48 |
| mycelium extract | 0.01 | 31 |
| | 0.005 | 17 |

TABLE 33

| | Concentration of | Cytotoxicity |
|---|---|---|
| Sample name | treated sample (wt %) | reduction (%) |
| Pleurotus ferulae | 0.02 | 41 |
| mycelium culture | 0.01 | 32 |
| | 0.005 | 16 |

As can be seen from measurement results (Tables 31 to 33) of reduction of cytotoxicity induced by ultraviolet irradiation, all of the Pleurotus ferulae fruiting body extract, the Pleurotus ferulae mycelium extract and the Pleurotus ferulae mycelium culture potently reduce cytotoxicity induced by ultraviolet radiation and thereby effectively inhibit induction of cytotoxicity by ultraviolet radiation.

From the results, it can be seen that the Pleurotus ferulae fruiting body extract, the Pleurotus ferulae mycelium extract and the Pleurotus ferulae mycelium culture are potently effective in reducing cytotoxicity induced by ultraviolet irradiation. This demonstrates that the composition for skin external application according to the present invention is potently effective in reducing skin irritation induced by ultraviolet radiation.

### Test Example 12

### Measurement of the Effect of Inhibition of Expression of Inflammatory Cytokine by Ultraviolet Irradiation

In the present test example, the inhibitory effects of the Pleurotus ferulae fruiting body extract obtained in Example 1, the Pleurotus ferulae mycelium extract obtained in Example 2 and the Pleurotus ferulae mycelium culture obtained in Example 3 on expression of inflammatory cytokine by ultraviolet irradiation were measured.

Fibroblasts isolated from human epidermal tissues were seeded at a density of 5×10⁴ on a 24-well test plate for 24 hours. Each well was washed with PBS once, 500 μl of PBS was added thereto. The fibroblasts were irradiated with 30 mJ/cm² of ultraviolet light using a ultraviolet B (UVB) lamp (Model: F15T8, UVB 15W, Sankyo Denki Co., Ltd., Japan), PBS was removed and 350 μl of a cell culture medium (DMEM not supplemented with 10% FBS) was added to the fibroblasts.

The fibroblasts were treated with 0.005, 0.01 and 0.02% by weight of the Pleurotus ferulae fruiting body extract, the Pleurotus ferulae mycelium extract and the Pleurotus ferulae mycelium culture and cultured for 5 hours. After culturing, 150 μl of a culture supernatant was collected and IL-1α was assayed to determine the effects of the Pleurotus ferulae fruiting body extract, the Pleurotus ferulae mycelium extract and the Pleurotus ferulae mycelium culture on repression of inflammatory cytokine. The amount of IL-1α was assayed by enzyme-linked immunosorbent assay (ELISA) and repression of inflammatory cytokine (%) was calculated in accordance with the following Equation 8.

Repression of inflammatory cytokine (%)=[1−(St−Bo)/(Bt−Bo)]×100 Equation 8

Bo: amount of IL-1α produced in well not irradiated with ultraviolet light and not treated with sample
Bt: amount of IL-1α produced in well irradiated with ultraviolet light and not treated with sample
St: amount of IL-1α produced in well irradiated with ultraviolet light and treated with sample

TABLE 34

| | Concentration of | Repression of |
|---|---|---|
| | treated sample | inflammatory |
| Sample name | (wt %) | cytokine (%) |
| Pleurotus ferulae | 0.02 | 34 |
| fruiting body extract | 0.01 | 28 |
| | 0.005 | 21 |

TABLE 35

| | Concentration of | Repression of |
|---|---|---|
| | treated sample | inflammatory |
| Sample name | (wt %) | cytokine (%) |
| Pleurotus ferulae | 0.02 | 32 |
| mycelium extract | 0.01 | 26 |
| | 0.005 | 18 |

TABLE 36

| | Concentration of | Repression of |
|---|---|---|
| | treated sample | inflammatory |
| Sample name | (wt %) | cytokine (%) |
| Pleurotus ferulae | 0.02 | 32 |
| mycelium culture | 0.01 | 25 |
| | 0.005 | 19 |

As can be seen from results (Tables 34 to 36) of measurement of inhibition of expression of inflammatory cytokine by ultraviolet irradiation, all of the Pleurotus ferulae fruiting body extract, the Pleurotus ferulae mycelium extract and the Pleurotus ferulae mycelium culture efficiently inhibit expression of inflammatory cytokine by ultraviolet radiation.

From the results, it can be seen that the Pleurotus ferulae fruiting body extract, the Pleurotus ferulae mycelium extract and the Pleurotus ferulae mycelium culture are potently effective in inhibiting expression of inflammatory cytokine by ultraviolet irradiation. This demonstrates that the composition for skin external application according to the present invention exhibits potent anti-inflammatory effects.

### Test Example 13

### Measurement of the Effect of Inhibiting Prostaglandin Biosynthesis by Ultraviolet Irradiation

In the present test example, the inhibitory effect of the Pleurotus ferulae fruiting body extract obtained in Example 1, the Pleurotus ferulae mycelium extract obtained in Example 2 and the Pleurotus ferulae mycelium culture obtained in Example 3 on prostaglandin (prostaglandin E₂; hereinafter, referred to “PGE₂”) biosynthesis was measured.

Keratinocytes isolated from human dermal tissues for the test were seeded at a density of 5×10⁴ on a 24-well test plate for 24 hours. The keratinocytes were cultured in a fresh medium containing no FBS for 18 hours, the keratinocytes were treated with aspirin such that a final concentration reached 50 μM to remove activity of prostaglandin biosynthesis enzymes present in keratinocytes (prostaglandin E₂ synthetase or cyclooxygenase, hereinafter referred to as “COX”). At two hours after treatment with aspirin, each well containing keratinocytes was washed with PBS twice and 100 μl of PBS was added to each well. The fibroblasts were irradiated with 10 mJ/cm² of ultraviolet light using a ultraviolet B (UVB) lamp (Model: F15T8, UVB 15W, Sankyo Denki Co., Ltd., Japan), PBS was removed and 250 fit of keratinocyte growth media (Clonetics, Biowhittaker Inc., MD, USA) was added to the respective wells. The fibroblasts were treated with 0.005, 0.01 and 0.02% by weight of the Pleurotus ferulae fruiting body extract, the Pleurotus ferulae mycelium extract and the Pleurotus ferulae mycelium culture and cultured for 16 hours. After culturing, a predetermined amount of culture supernatant was collected and PGE₂ (prostaglandin E₂) bio-synthesized for 16 hours was assayed to determine the inhibitory effect of the Pleurotus ferulae fruiting body extract, the Pleurotus ferulae mycelium extract and the Pleurotus ferulae mycelium culture on prostaglandin biosynthesis. The PGE₂ was assayed by enzyme-linked immunosorbent assay (ELISA).

TABLE 37

| | Inhibition of |
|---|---|
| Sample name | PGE₂ (%) |
| (−)UV B | Control |
| (+)UV B | — |
| (+)UV B + Pleurotus ferulae fruiting | 55 |
| body extract 0.02 wt % | |
| (+)UV B + Pleurotus ferulae fruiting | 31 |
| body extract 0.01 wt % | |
| (+)UV B + Pleurotus ferulae fruiting | 12 |
| body extract 0.005 wt % | |

TABLE 38

| Sample name | Inhibition of PGE₂ (%) |
|---|---|
| (−)UV B | Control |
| (+)UV B | — |
| (+)UV B + Pleurotus ferulae mycelium | 56 |
| extract 0.02% by weight | |
| (+)UV B + Pleurotus ferulae mycelium | 31 |
| extract 0.01% by weight | |
| (+)UV B + Pleurotus ferulae mycelium | 13 |
| extract 0.005% by weight | |

TABLE 39

| Sample name | Inhibition of PGE₂ (%) |
|---|---|
| (−)UV B | Control |
| (+)UV B | — |
| (+)UV B + Pleurotus ferulae mycelium | 53 |
| culture 0.02% by weight | |
| (+)UV B + Pleurotus ferulae mycelium | 29 |
| culture 0.01% by weight | |
| (+)UV B + Pleurotus ferulae mycelium | 11 |
| culture 0.005% by weight | |

As can be seen from results (Tables 37 to 39) of measurement of inhibition on prostaglandin biosynthesis by ultraviolet irradiation, all of the Pleurotus ferulae fruiting body extract, the Pleurotus ferulae mycelium extract and the Pleurotus ferulae mycelium culture efficiently inhibit prostaglandin biosynthesis by ultraviolet irradiation. In particular, the PGE₂ is known to be predominantly produced by COX-2 enzymes. As such, it can be seen from the test that the Pleurotus ferulae fruiting body extract, the Pleurotus ferulae mycelium extract and the Pleurotus ferulae mycelium culture inhibit induction action or activity of COX-2 enzymes.

From the results, it can be seen that the Pleurotus ferulae fruiting body extract, the Pleurotus ferulae mycelium extract and the Pleurotus ferulae mycelium culture are potently effective in inhibiting prostaglandin biosynthesis by ultraviolet irradiation. This demonstrates that the composition for skin external application according to the present invention is potently effective inhibiting prostaglandin biosynthesis.

### Test Example 14

### Measurement of Antibacterial Activity Against Acne Bacteria

In the present test example, antibacterial activity against acne bacteria of the Pleurotus ferulae fruiting body extract obtained in Example 1, the Pleurotus ferulae mycelium extract obtained in Example 2 and the Pleurotus ferulae mycelium culture obtained in Example 3 was measured using paper disc test.

First, propionibacterium acnes, skin flora causing acne were subcultured in a BHI liquid medium (brain-heart infusion broth; 3.7%) for 48 hours to activate the propionibacterium acnes. 0.1 mL of the bacterial culture thus prepared was smeared on BHI solid media (brain-heart infusion broth; 3.7%, agar; 1.5%), followed by drying. Each of the Pleurotus ferulae fruiting body extract, the

Pleurotus ferulae mycelium extract and the Pleurotus ferulae mycelium culture was diluted at 12% (w/v) with a 95% aqueous ethanol solution, 50 μl of the dilution was added dropwise to a paper disc with a diameter of 8 mm and the paper disc placed on the previously prepared solid medium was cultured in a 35° C. anaerobic incubator for 3 days. A bacterial growth zone created around the paper disc was observed and a size of the inhibition zone was measured to evaluate antibacterial activity.

As a result of measurement of antibacterial activity against propionibacterium acnes, the size of bacterial growth inhibition zone associated with the Pleurotus ferulae fruiting body extract, the Pleurotus ferulae mycelium extract and the Pleurotus ferulae mycelium culture was 16 mm, which means that the composition for skin external application of the present invention exhibits superior antibacterial activity.

### Test Example 15

### Measurement of Anti-Inflammatory Effect of Pleurotus ferulae Fruiting Body Extract, Pleurotus ferulae Mycelium Extract and Pleurotus ferulae Mycelium Culture after Occurrence of Atopic Dermatitis by Compound 48/80

In the present test example, the anti-inflammatory effects of the Pleurotus ferulae fruiting body extract obtained in Example 1, the Pleurotus ferulae mycelium extract obtained in Example 2 and the Pleurotus ferulae mycelium culture obtained in Example 3 after induction of atopic dermatitis by Compound 48/80 were measured.

30 μl of a compound 48/80 (Sigma, Co.; 1 mg/mL in saline) was injected into the dermis of the dorsal neck skin of BALB/c mice (5 wks, male) and behaviors the mice scratched the back of the neck in a cage were observed for 60 hours. In order to identify anti-inflammation efficacy of the Pleurotus ferulae fruiting body extract, the Pleurotus ferulae mycelium extract and the Pleurotus ferulae mycelium culture, 100 μl (250 μl/mL) of the Pleurotus ferulae fruiting body extract, the Pleurotus ferulae mycelium extract and the Pleurotus ferulae mycelium culture were applied to the dermis of the dorsal neck skin of each mouse starting 5 days before injection of the compound 48/80. The Pleurotus ferulae fruiting body extract, the Pleurotus ferulae mycelium extract and the Pleurotus ferulae mycelium culture were applied to the dermis of the dorsal neck skin of each mouse 10 times in total twice a day for 5 days to perform pre-treatment, the compound 48/80 was injected into the dermis, the time for which the mouse scratched the neck with the hind legs was measured at 5-minute intervals for 75 minutes in total, and a degree of pruritus was evaluated.

TABLE 40

| | Number of scratching (in 5 min) | | | | | | | | | | | |
|---|---|---|---|---|---|---|---|---|---|---|---|---|
| Sample name | 5 | 10 | 15 | 20 | 25 | 30 | 35 | 40 | 45 | 50 | 55 | 60 |
| Pleurotus | 22 | 24 | 28 | 27 | 29 | 34 | 36 | 38 | 41 | 23 | 14 | 12 |
| ferulae | | | | | | | | | | | | |
| fruiting body | | | | | | | | | | | | |
| extract | | | | | | | | | | | | |
| Control group | 39 | 41 | 50 | 62 | 88 | 94 | 98 | 72 | 59 | 50 | 40 | 32 |

TABLE 41

| | Number of scratching (in 5 min) | | | | | | | | | | | |
|---|---|---|---|---|---|---|---|---|---|---|---|---|
| Sample name | 5 | 10 | 15 | 20 | 25 | 30 | 35 | 40 | 45 | 50 | 55 | 60 |
| Pleurotus | 21 | 23 | 26 | 27 | 29 | 33 | 35 | 38 | 40 | 21 | 11 | 9 |
| ferulae | | | | | | | | | | | | |
| mycelium | | | | | | | | | | | | |
| extract | | | | | | | | | | | | |
| Control group | 39 | 41 | 50 | 62 | 88 | 94 | 98 | 72 | 59 | 50 | 40 | 32 |

TABLE 42

| | Number of scratching (in 5 min) | | | | | | | | | | | |
|---|---|---|---|---|---|---|---|---|---|---|---|---|
| Sample name | 5 | 10 | 15 | 20 | 25 | 30 | 35 | 40 | 45 | 50 | 55 | 60 |
| Pleurotus | 21 | 23 | 25 | 28 | 27 | 32 | 35 | 39 | 40 | 22 | 13 | 10 |
| ferulae | | | | | | | | | | | | |
| mycelium | | | | | | | | | | | | |
| culture | | | | | | | | | | | | |
| Control group | 39 | 41 | 50 | 62 | 88 | 94 | 98 | 72 | 59 | 50 | 40 | 32 |

As can be seen from results (Tables 40 to 42) of measurement of anti-inflammatory effects of the Pleurotus ferulae fruiting body extract, the Pleurotus ferulae mycelium extract and the Pleurotus ferulae mycelium after occurrence of atopic dermatitis, all of the Pleurotus ferulae fruiting body extract, the Pleurotus ferulae mycelium extract and the Pleurotus ferulae mycelium culture inhibit induction of atopic dermatitis (skin pruritus) by the compound 48/80.

From the results, it can be seen that the Pleurotus ferulae fruiting body extract, the Pleurotus ferulae mycelium extract and the Pleurotus ferulae mycelium culture are potently effective in preventing atopy. This demonstrates that the composition for skin external application according to the present invention is potently effective in alleviating, preventing or treating atopy.

### Test Example 16

### Measurement of Hygroscopicity

In the present test example, hygroscopicity of the Pleurotus ferulae fruiting body extract obtained in Example 1, the Pleurotus ferulae mycelium extract obtained in Example 2 and the Pleurotus ferulae mycelium culture obtained in Example 3 was measured.

The Pleurotus ferulae fruiting body extract, the Pleurotus ferulae mycelium extract and the Pleurotus ferulae mycelium culture, and distilled water were saturated in dried human epidermal cells, an amount of absorbed water was measured, and variation in weight after 48 hours was measured. Amounts of water absorbed per unit weight of skin cells were compared based on the respective variations in measured weights. This test is used as a method for comparing amounts absorbed in epidermal cells and for estimating moisturizing effects on the human skin.

Specifically, human epidermal cells were dried, an amount of moisture contained per unit weight of the cells was adjusted to a predetermined level, and weights of respective epidermal cell samples and amounts of contained water were measured using a Kaiser's method. After measurement of water content, the epidermal cells were immersed and satarably absorbed in the Pleurotus ferulae fruiting body extract, the Pleurotus ferulae mycelium extract, the Pleurotus ferulae mycelium culture, and purified water for 24 hours, the epidermal cells in which water was saturated (completely absorbed), were weighed, and a predetermined amount of the epidermal cells was harvested and measured for an amount of moisture per unit weight of the epidermal cells using a Kaiser moisture gauge. Then, the epidermal cells were dried under reduced pressure for 48 hours and weighed, and a predetermined amount of the epidermal cells was harvested and measured for an amount of moisture per unit weight of the epidermal cells using a Kaiser moisture gauge (weight of moisture (mg)/weight (g) of epidermal cell).

TABLE 43

| | Moisture content | | |
|---|---|---|---|
| | Initially | | |
| Sample name | dried | Saturated | Re-dried |
| Pleurotus ferulae | 400 | 790 | 610 |
| fruiting body extract | | | |
| Purified water | 400 | 780 | 400 |

TABLE 44

| | Moisture content | | |
|---|---|---|---|
| | Initially | | |
| Sample name | dried | Saturated | Re-dried |
| Pleurotus ferulae | 400 | 800 | 620 |
| mycelium extract | | | |
| Purified water | 400 | 780 | 400 |

TABLE 45

| | Moisture content | | |
|---|---|---|---|
| | Initially | | |
| Sample name | dried | Saturated | Re-dried |
| Pleurotus ferulae | 400 | 820 | 630 |
| mycelium culture | | | |
| Purified water | 400 | 780 | 400 |

As can be seen from results (Tables 43 to 45) of measurement of hygroscopicity of the Pleurotus ferulae fruiting body extract, the Pleurotus ferulae mycelium extract and the Pleurotus ferulae mycelium culture, the epidermal cells saturated with the Pleurotus ferulae fruiting body extract, the Pleurotus ferulae mycelium extract and the Pleurotus ferulae mycelium culture contain much more water during re-drying than epidermal cells saturated with purified water.

From the results, it can be seen that the Pleurotus ferulae fruiting body extract, the Pleurotus ferulae mycelium extract and the Pleurotus ferulae mycelium culture exhibits potent hygroscopicity. This demonstrates that the composition for skin external application according to the present invention exhibit potent moisturizing effect.

### Test Example 17

### Measurement of the Effect of Binding to Human Hair Protein

In the present test example, the effects of binding to human hair protein of the Pleurotus ferulae fruiting body extract obtained in Example 1, the Pleurotus ferulae mycelium extract obtained in Example 2 and the Pleurotus ferulae mycelium culture obtained in Example 3 were measured.

Test concentrations of 0.5, 0.25 and 0.1% by weight of the Pleurotus ferulae fruiting body extract, the Pleurotus ferulae mycelium extract and the Pleurotus ferulae mycelium culture were reacted with 50 μg of human hair keratin protein at room temperature for 30 minutes, and reacted with a solution (1:1) of 6% hydrogen peroxide and 0.5% ammonia for 30 minutes. The reaction solution was electrophoresed on a 10% SDS-polyacrylamide gel in accordance with the Laemmli's method, keratin protein present on the gel was dyed with 0.1% Coomassie brilliant blue R 250 in the presence of a mixed solution of 10% glacial acetic acid and 40% ethanol for one hour, decolorized with a mixed solution of 10% glacial acetic acid and 40% ethanol and a keratin protein band was confirmed. Using the imagemaster software package (Amersham Pharmacia Biotech.), keratin binding capacity (%) was expressed with respect to 100% of a keratin protein band appearing after electrophoresis of only 50 μg of human hair keratin protein for, as a control group, a group not treated with the Pleurotus ferulae fruiting body extract, the Pleurotus ferulae mycelium extract or the Pleurotus ferulae mycelium culture.

TABLE 46

| Pleurotus ferulae | Control | | | |
|---|---|---|---|---|
| fruiting body extract | group | 0.5 wt % | 0.25 wt % | 0.1 wt % |
| Binding capacity to | 100 | 152 | 139 | 121 |
| hair protein | | | | |

TABLE 47

| Pleurotus ferulae | Control | | | |
|---|---|---|---|---|
| mycelium extract | group | 0.5 wt % | 0.25 wt % | 0.1 wt % |
| Binding capacity to | 100 | 151 | 138 | 120 |
| hair protein | | | | |

TABLE 48

| Pleurotus ferulae | Control | | | |
|---|---|---|---|---|
| mycelium culture | group | 0.5 wt % | 0.25 wt % | 0.1 wt % |
| Binding capacity to | 100 | 148 | 136 | 119 |
| hair protein | | | | |

It can be seen from test results (Tables 46 to 48) that all of the Pleurotus ferulae fruiting body extract, the Pleurotus ferulae mycelium extract and the Pleurotus ferulae mycelium culture exhibit an increase in binding capacity to hair protein keratins as content thereof increases.

### Test Example 18

### Measurement of Blocking Effect of Pleurotus ferulae Fruiting Body Extract and Pleurotus ferulae Mycelium Culture on Release of Keratin by Treatment with Alkali and Hydrogen Peroxide

In the present test example, the blocking effects of the Pleurotus ferulae fruiting body extract obtained in Example 1, the Pleurotus ferulae mycelium extract obtained in Example 2 and the Pleurotus ferulae mycelium culture obtained in Example 3 on release of keratin by treatment with alkali and hydrogen peroxide were measured.

3 g of hair was added to 10 mL of a mixed solution containing hydrogen peroxide (6%) and ammonia (1.68%) in a ratio of 1:1, and the hair solution was treated with test concentrations of 0.5, 0.25 and 0.1% by weight of the Pleurotus ferulae fruiting body extract, the Pleurotus ferulae mycelium extract and the Pleurotus ferulae mycelium culture at room temperature for 30 minutes. 0.5 mL of the reaction solution was concentrated with a rapid-con protein concentration kit (Elpis Biotech.), the concentrate was electrophoresed on a 10% SDS-polyacrylamide gel in accordance with the Laemmli method, keratin proteins present on the gel was dyed with 0.1% Coomassie brilliant blue R 250 in the presence of a mixed solution of 10% glacial acetic acid and 40% ethanol for one hour and decolorized with a mixed solution of 10% glacial acetic acid and 40% ethanol to confirm a keratin protein band. Using the imagemaster software package (Amersham Pharmacia Biotech.), keratin binding capacity (%) was expressed with respect to 100% of a keratin protein band appearing after electrophoresis of only 50 μg of human hair keratin protein for, as a control group, a group not treated with the Pleurotus ferulae fruiting body extract, the Pleurotus ferulae mycelium extract or the Pleurotus ferulae mycelium culture.

TABLE 49

| Pleurotus ferulae fruiting | Control | | | |
|---|---|---|---|---|
| body extract | group | 0.5 wt % | 0.25 wt % | 0.1 wt % |
| Keratin protein released | 100 | 39 | 51 | 76 |
| from hair | | | | |

TABLE 50

| Pleurotus ferulae mycelium | Control | | | |
|---|---|---|---|---|
| extract | group | 0.5 wt % | 0.25 wt % | 0.1 wt % |
| Keratin protein released | 100 | 37 | 49 | 75 |
| from hair | | | | |

TABLE 51

| Pleurotus ferulae mycelium | Control | | | |
|---|---|---|---|---|
| culture | group | 0.5 wt % | 0.25 wt % | 0.1 wt % |
| Keratin protein released | 100 | 33 | 49 | 72 |
| from hair | | | | |

It can be seen from test results (Tables 49 to 51) that all of the Pleurotus ferulae fruiting body extract, the Pleurotus ferulae mycelium extract and the Pleurotus ferulae mycelium culture exhibit a decrease in amounts of keratin proteins released from hairs as content thereof increases.

### Test Example 19

### Measurement of Effect of Pleurotus ferulae Fruiting Body Extract and Pleurotus ferulae Mycelium Culture on Tensile Strength and Elongation of Hairs by Treatment with Alkali and Hydrogen Peroxide

In the present test example, the effects of the Pleurotus ferulae fruiting body extract obtained in Example 1, the Pleurotus ferulae mycelium extract obtained in Example 2 and the Pleurotus ferulae mycelium culture obtained in Example 3 on tensile strength and elongation of hairs by treatment with alkali and hydrogen peroxide were measured.

3 g of hairs were added to 10 mL of a mixed solution containing hydrogen peroxide (6%) and ammonia (1.68%) at a ratio of 1:1, the hair solution was treated with test concentrations of 0.5, 0.25 and 0.1% by weight of the Pleurotus ferulae fruiting body extract, the Pleurotus ferulae mycelium extract and the Pleurotus ferulae mycelium culture at room temperature for 30 minutes, washed with water and dried in air. Then, tensile strength (gf) and elongation (%) of the hair solution were measured using an autograph tester.

TABLE 52

| Pleurotus ferulae fruiting | Purified | | | |
|---|---|---|---|---|
| body extract | water | 0.5 wt % | 0.25 wt % | 0.1 wt % |
| Tensile strength (gf) | 112 | 125 | 121 | 117 |
| Elongation (%) | 46 | 51 | 50 | 48 |

TABLE 53

| Pleurotus ferulae mycelium | Purified | | | |
|---|---|---|---|---|
| extract | water | 0.5 wt % | 0.25 wt % | 0.1 wt % |
| Tensile strength (gf) | 112 | 126 | 122 | 119 |
| Elongation (%) | 46 | 52 | 50 | 48 |

TABLE 54

| Pleurotus ferulae mycelium | Purified | | | |
|---|---|---|---|---|
| culture | water | 0.5 wt % | 0.25 wt % | 0.1 wt % |
| Tensile strength (gf) | 112 | 123 | 121 | 118 |
| Elongation (%) | 46 | 52 | 49 | 47 |

It can be seen from test results (Tables 52 to 54) that the Pleurotus ferulae fruiting body extract, the Pleurotus ferulae mycelium extract and the Pleurotus ferulae mycelium culture inhibit deterioration in strength of hairs as content thereof increases when the hairs are treated with a decolorizing agent (the mixed solution of hydrogen peroxide and ammonia) for 30 minutes. Accordingly, the Pleurotus ferulae fruiting body extract, the Pleurotus ferulae mycelium extract and the Pleurotus ferulae mycelium culture have the effect of protecting hairs treated with a decolorizing agent.

### Test Example 20

### Hair Growth Effect Test

In the present test example, the hair growth effect of the Pleurotus ferulae fruiting body extract obtained in Example 1, the Pleurotus ferulae mycelium extract obtained in Example 2 and the Pleurotus ferulae mycelium culture obtained in Example 3 was identified.

30% aqueous ethanol solutions containing 2% by weight of the Pleurotus ferulae fruiting body extract, the Pleurotus ferulae mycelium extract and the Pleurotus ferulae mycelium culture were respectively prepared for use in the hair growth effect test. The hair growth effect test was performed on 47 to 53 day old mice (ICR). Hairs on the back of the mice were removed, 10 mice having the back where the hairs were clearly removed were grouped for respective substance groups and 100 mL of the sample was applied to each mouse daily starting the following day of the test. Hair length and hair growth with the passage of time were scored from 0 to 2 according to hair recovery after removal of hairs and compared. In order to compare hair growth, growth of a group wherein a 30% alcohol solution was applied to each mouse as a control group was observed.

In addition, facilitation effect on hair growth was evaluated on a 3-grade scale (0: no growth, 1: slight growth, 2: substantial growth).

TABLE 55

| | number of passed days | | |
|---|---|---|---|
| Sample | 5 | 10 | 15 |
| Pleurotus ferulae fruiting body extract 2% | 0.14 | 0.63 | 1.39 ± 0.07 |
| by weight | | | |
| Control group | 0.07 | 0.13 | 0.80 ± 0.24 |

TABLE 56

| | number of passed days | | |
|---|---|---|---|
| Sample | 5 | 10 | 15 |
| Pleurotus ferulae mycelium | 0.15 | 0.68 | 1.39 ± 0.04 |
| extract 2% by weight | | | |
| Control group | 0.07 | 0.13 | 0.80 ± 0.24 |

TABLE 57

| | number of passed days | | |
|---|---|---|---|
| Sample | 5 | 10 | 15 |
| Pleurotus ferulae mycelium | 0.16 | 0.71 | 1.41 ± 0.08 |
| culture 2% by weight | | | |
| Control group | 0.07 | 0.13 | 0.80 ± 0.24 |

It can be seen from test results (Tables 55 to 57) that the Pleurotus ferulae fruiting body extract, the Pleurotus ferulae mycelium extract and the Pleurotus ferulae mycelium culture are potently effective in facilitating hair growth.

### Example 4

### Preparation of Cosmetic Containing Pleurotus ferulae Fruiting Body Extract

In the present example, a cosmetic containing the Pleurotus ferulae fruiting body extract obtained in Example 1 was prepared.

The prepared cosmetic was provided as a cream and the composition thereof is shown in the following Table 58.

First, the phase (A) described in the following Table 58 was heated and stored at 70° C., the phase (A) was then added to the phase (B) to perform preliminary emulsification, and the resulting emulsion was homogeneously main-emulsified using a homomixer and slowly cooled to prepare a cream.

TABLE 58

| | |
|---|---|
| Raw materials | Example 4 (wt %) |

| | | |
|---|---|---|
| A | Stearyl alcohol | 8 |
| | Stearic acid | 2 |
| | Cholesteryl stearate | 2 |
| | Squalane | 4 |
| | 2-octyldodecyl alcohol | 6 |
| | Polyoxyethylene (25 mol- | 3 |
| | added) alcohol ester | |
| | Glyceryl monostearate ester | 2 |
| B | Pleurotus ferulae fruiting body extract | 1 |
| | Propylene glycol | 5 |
| | Purified water | Balance with |
| | | respect to 100 |
| | | in total |

### Example 5

### Preparation of Cosmetic Containing Pleurotus ferulae Mycelium Extract

In the present example, a cosmetic containing the Pleurotus ferulae mycelium extract obtained in Example 2 was prepared.

The prepared cosmetic was provided as a cream and the composition thereof is shown in the following Table 59.

First, the phase (A) described in the following Table 58 was heated and stored at 70° C., the phase (A) was then added to the phase (B) to perform preliminary emulsification, and the resulting emulsion was homogeneously main-emulsified using a homomixer and slowly cooled to prepare a cream.

TABLE 59

| Raw material | | Example 5 (wt %) |
|---|---|---|
| A | Stearyl alcohol | 8 |
| | Stearic acid | 2 |
| | Cholesteryl stearate | 2 |
| | Squalane | 4 |
| | 2-octyldodecyl alcohol | 6 |
| | Polyoxyethylene (25 mol- | 3 |
| | added)alcohol ester | |
| | Glyceryl monostearate ester | 2 |
| B | Pleurotus ferulae mycelium extract | 1 |
| | Propylene glycol | 5 |
| | Purified water | Balance with |
| | | respect to 100 |
| | | in total |

### Example 6

### Preparation of Cosmetic Containing Pleurotus ferulae Mycelium Culture

In the present example, a cosmetic containing the Pleurotus ferulae mycelium culture obtained in Example 3 was prepared.

The prepared cosmetic was provided as a cream and the composition thereof is shown in the following Table 60.

First, the phase (A) described in the following Table 60 was heated and stored at 70° C., the phase (A) was then added to the phase (B) to perform preliminary emulsification, and the resulting emulsion was homogeneously main-emulsified using a homomixer and slowly cooled to prepare a cream.

TABLE 60

| Raw materials | | Example 6 (wt %) |
|---|---|---|
| A | Stearyl alcohol | 8 |
| | Stearic acid | 2 |
| | Cholesteryl stearate | 2 |
| | Squalane | 4 |
| | 2-Octyldodecyl alcohol | 6 |
| | Polyoxyethylene (25 mol- | 3 |
| | added)alcohol ester | |
| | Glyceryl monostearate ester | 2 |
| B | Pleurotus ferulae mycelium culture | 1 |
| | Propylene glycol | 5 |
| | Purified water | Balance with |
| | | respect to 100 |
| | | in total |

### Comparative Example 1

### Preparation of General Cosmetic

In the present comparative example, a general cosmetic not containing the Pleurotus ferulae fruiting body extract obtained in Example 1, the Pleurotus ferulae mycelium extract obtained in Example 2 and the Pleurotus ferulae mycelium culture obtained in Example 3 was prepared.

The prepared cosmetic was provided as a cream and the composition thereof is shown in the following Table 61.

First, the phase (A) described in the following Table 61 was heated and stored at 70° C., the phase (A) was then added to the phase (B) to perform preliminary emulsification, and the resulting emulsion was homogeneously main-emulsified using a homomixer and slowly cooled to prepare a cream.

TABLE 61

| | | Comparative |
|---|---|---|
| Raw material | | Example 1 (wt %) |
| A | Stearyl alcohol | 8 |
| | Stearic acid | 2 |
| | Cholesteryl stearate | 2 |
| | Squalane | 4 |
| | 2-Octyldodecyl alcohol | 6 |
| | Polyoxyethylene (25 mol- | 3 |
| | added)alcohol ester | |
| | Glyceryl monostearate ester | 2 |
| B | Pleurotus ferulae fruiting body | — |
| | extract or Pleurotus ferulae | |
| | mycelium extract or Pleurotus | |
| | ferulae mycelium culture | |
| | Propylene glycol | 5 |
| | Purified water | Balance with |
| | | respect to 100 |
| | | in total |

### Test Example 21

### Measurement of the Effect of Cosmetic on Improvement of Skin Elasticity

In order to examine the effect of cosmetics prepared in Examples 4 to 6 and Comparative Example 1 on improvement of skin elasticity, the cosmetics prepared in Examples 4 to 6 and the cosmetic prepared in Comparative Example 1 were continuously applied to the right and left sides, respectively, of the face of 20 subjects (20 to 35 year old females) twice a day for two months.

In order to identify the effect of improving skin elasticity, skin elasticity was measured using a skin elasticity meter (cutometer SEM 575, C+K Electronic Co., Germany) before use of products and after use thereof for two months. Test results are shown as ΔR7 of Cutometer SEM 575 in the following Tables 62 to 64. R7 represents viscoelasticity of skin (n=20, p<0.05).

TABLE 62

| Test product | Skin elasticity (R7) |
|---|---|
| Example 4 | 0.25 |
| Comparative | 0.11 |
| Example 1 | |

TABLE 63

| Test product | Skin elasticity (R7) |
|---|---|
| Example 5 | 0.22 |
| Comparative | 0.11 |
| Example 1 | |

TABLE 64

| Test product | Skin elasticity (R7) |
|---|---|
| Example 6 | 0.23 |
| Comparative | 0.11 |
| Example 1 | |

As can be seen from results (Tables 62 to 64) of the test of the effect of cosmetics on improvement of skin elasticity, subjects to whom Example 4 containing the Pleurotus ferulae fruiting body extract, Example 5 containing the Pleurotus ferulae mycelium extract, and Example 6 containing the Pleurotus ferulae mycelium culture are applied show better skin elasticity improvement than subjects to whom Comparative Example 1 is applied.

From the results, it can be seen that the Pleurotus ferulae fruiting body extract, the composition for skin external application according to the present invention is potently effective in improving skin elasticity.

### Test Example 22

### Measurement of the Effect of Cosmetic on Alleviation of Skin Wrinkles

In order to examine the effect of cosmetics prepared in Examples 4 to 6 and Comparative Example 1 on alleviation of skin wrinkle, the cosmetics prepared in Examples 4 to 6 and the cosmetic prepared in Comparative Example 1 were continuously applied to the right and left sides, respectively, of the face of 20 subjects (20 to 35 year old females) twice a day for two months.

In order to identify skin wrinkle alleviation, replicas made of silicon were fabricated before use of products and after use of products for two months and states of wrinkles in predetermined sites were observed using visiometer (SV60, C+K Electronic Co., Germany).

The measurement results are shown in Tables 65 and 67 below and are expressed as the averages of values obtained by subtracting parameter values after two months from parameter values before two months. In other words, more negative average values indicate better alleviation of wrinkles.

TABLE 65

| Test product | R1 | R2 | R3 | R4 | R5 |
|---|---|---|---|---|---|
| Example 4 | −0.22 | −0.18 | −0.12 | −0.09 | −0.07 |
| Comparative | −0.12 | −0.09 | −0.06 | −0.06 | −0.04 |
| Example 1 | | | | | |

R1: difference between the peak and bottom of a wrinkle contour line
R2: average of R1 values of five sections randomly divided from the wrinkle contour line
R3: maximum of R1 values of five sections
R4: average of values obtained by subtracting the values of the peak and valley from the baseline of the wrinkle contour line
R5: average of values obtained by subtracting a wrinkle outline from the baseline of the wrinkle contour line

TABLE 66

| Test product | R1 | R2 | R3 | R4 | R5 |
|---|---|---|---|---|---|
| Example 5 | −0.20 | −0.17 | −0.11 | −0.07 | −0.05 |
| Comparative | −0.12 | −0.09 | −0.06 | −0.06 | −0.04 |
| Example 1 | | | | | |

R1: difference between the peak and bottom of a wrinkle contour line
R2: average of R1 values of five sections randomly divided from the wrinkle contour line
R3: maximum of R1 values of five sections
R4: average of values obtained by subtracting the values of the peak and valley from the baseline of the wrinkle contour line
R5: average of values obtained by subtracting a wrinkle outline from the baseline of the wrinkle contour line

TABLE 67

| Test product | R1 | R2 | R3 | R4 | R5 |
|---|---|---|---|---|---|
| Example 6 | −0.21 | −0.16 | −0.11 | −0.07 | −0.06 |
| Comparative | −0.11 | −0.09 | −0.06 | −0.05 | −0.03 |
| Example 1 | | | | | |

R1: difference between the peak and bottom of a wrinkle contour line
R2: average of R1 values of five sections randomly divided from the wrinkle contour line
R3: maximum of R1 values of five sections
R4: average of values obtained by subtracting the values of the peak and valley from the baseline of the wrinkle contour line
R5: average of values obtained by subtracting a wrinkle outline from the baseline of the wrinkle contour line

It can be seen from results (Tables 65 to 67) of the test of the effect of the cosmetics on alleviation of skin wrinkle that Example 4 containing the Pleurotus ferulae fruiting body extract, Example 5 containing the Pleurotus ferulae mycelium extract, and Example 6 containing the Pleurotus ferulae mycelium culture show better skin wrinkle alleviation than Comparative Example 1.

From the results, it can be seen that the Pleurotus ferulae fruiting body extract, the composition for skin external application according to the present invention is potently effective in alleviating skin wrinkle.

### Test Example 23

### Measurement of Whitening Effect of Cosmetic

In the test Example, whitening effects of the cosmetics prepared in Example 4 to 6 and Comparative Example 1 were measured.

The cosmetics prepared in Examples 4 to 6 and the cosmetic prepared in Comparative Example 1 were continuously applied to the right and left sides, respectively, of the face of 20 subjects (20 to 35 year old females) twice a day for two months.

After two months, skin sites at left and right sides of the face to which the cosmetics were applied were observed using a visiometer, change in brightness of skin tone (ΔL) was measured using a chromameter (Minolta CR300), objective visual inspection by a plurality of skilled persons and subjective visual inspection by the subjects were performed and the whitening effect was evaluated according to the following grade. The results are shown in the following Tables 68 to 70 and whitening effect were evaluated on a 7-grade scale (−3: considerably aggravated, −2: aggravated, −1: slightly aggravated, 0: not changed, 1: slightly alleviated, 2: alleviated, 3: considerably alleviated).

TABLE 68

| | | | Objective visual | | Subjective visual | |
|---|---|---|---|---|---|---|
| | Change in skin tone | | inspection by | | inspection by | |
| | brightness (ΔL) | | skilled persons | | subjects | |
| | Example | Comparative | Example | Comparative | Example | Comparative |
| | 4 | Example 1 | 4 | Example 1 | 4 | Example 1 |
| Average | 3.3 | 1.8 | 2.9 | 1.9 | 2.6 | 1.3 |

TABLE 69

| | | | Objective visual | | Subjective visual | |
|---|---|---|---|---|---|---|
| | Change in skin tone | | inspection by | | inspection by | |
| | brightness (ΔL) | | skilled persons | | subjects | |
| | Example | Comparative | Example | Comparative | Example | Comparative |
| | 5 | Example 1 | 5 | Example 1 | 5 | Example 1 |
| Average | 3.2 | 1.8 | 2.8 | 2.0 | 2.5 | 1.4 |

TABLE 70

| | | | Objective visual | | Subjective visual | |
|---|---|---|---|---|---|---|
| | Change in skin tone | | inspection by | | inspection by | |
| | brightness (ΔL) | | skilled persons | | subjects | |
| | Example | Comparative | Example | Comparative | Example | Comparative |
| | 6 | Example 1 | 6 | Example 1 | 6 | Example 1 |
| Average | 3.2 | 1.7 | 2.8 | 2.0 | 2.4 | 1.4 |

It can be seen from results (Tables 68 to 70) of the test of whitening effect of the cosmetics that Example 4 containing the Pleurotus ferulae fruiting body extract, Example 5 containing the Pleurotus ferulae mycelium extract, and Example 6 containing the Pleurotus ferulae mycelium culture exhibit better whitening effects than Comparative Example 1.

From the results, it can be seen that the composition for skin external application according to the present invention is potently effective in whitening skin.

### Test Example 24

### Measurement of Test of the Effect of Cosmetic on Alleviation of Atopic Dermatitis

In the test Example, the effect of the cosmetics prepared in Example 4 to 6 and Comparative Example 1 on alleviation of atopic dermatitis was measured.

5 to 30 year old patients (n=30), who had suffered from atopic dermatitis for two years or longer, were tested for alleviation of atopic dermatitis. Example 4 or 6 and Comparative Example 1 were applied to the skin of the left and right hands, respectively, of each patient after cleansing every night once a day for 60 days, and the alleviation degrees of atopic dermatitis symptoms were tested. The testing was carried out by sensory evaluation using a questionnaire.

TABLE 71

| | Very good | Good | Not good |
|---|---|---|---|
| Example 4 | 80% (n = 24) | 20% (n = 6) | 0% |
| Comparative | 10% (n = 3) | 20% (n = 6) | 70% (n = 21) |
| Example 1 | | | |

TABLE 72

| | Very good | Good | Not good |
|---|---|---|---|
| Example 5 | 70% (n = 21) | 30% (n = 9) | 0% |
| Comparative | 10% (n = 3) | 20% (n = 6) | 70% (n = 21) |
| Example 1 | | | |

TABLE 73

| | Very good | Good | Not good |
|---|---|---|---|
| Example 6 | 70% (n = 21) | 30% (n = 9) | 0% |
| Comparative | 10% (n = 3) | 20% (n = 6) | 70% (n = 21) |
| Example 1 | | | |

It can be seen from results (Tables 71 to 73) of the test of the effect of the cosmetics on alleviation of atopic dermatitis that Example 4 containing the Pleurotus ferulae fruiting body extract, Example 5 containing the Pleurotus ferulae mycelium extract, and Example 6 containing the Pleurotus ferulae mycelium culture are more effective in alleviating atopic dermatitis than Comparative Example 1.

From the results, it can be seen that the composition for skin external application according to the present invention is potently effective in alleviating atopic dermatitis.

### Test Example 25

### Measurement of Effect of Cosmetic on Reduction of Skin Irritation by SLS

In the present test example, the effect of cosmetics prepared in Example 4 to 6 on reduction of skin irritation was tested by a human patch test.

A mixture of 1% of sodium lauryl sulfate (SLS) causing irritation on a general cosmetic formulation (cream, lotion, skin and essence) and the cosmetic of Example 4 or 6 was patched for 24, 48 and 72 hours, and irritation reduction was evaluated based on irritation induction index.

At 24 hours after 0.3 mg of the product was patched to the upper arm of 20 to 50 years old healthy males and females (n=50) using a Finn chamber (Finland), acute irritation index was evaluated. After evaluation, the same amount of product was patched to the same site. Delayed irritation index after 48 hours and 72 hours was evaluated.

As a result of the test of the effect of reducing skin irritation by SLS, the site to which only SLS was patched showed reddish irritation, while Example 4 containing the Pleurotus ferulae fruiting body extract, Example 5 containing the Pleurotus ferulae mycelium extract and Example 6 containing the Pleurotus ferulae mycelium culture did not show any skin irritation even after 24 hours, 48 hours and 72 hours.

From the results, it can be seen that the Pleurotus ferulae fruiting body extract, the cosmetics employing a combination of the Pleurotus ferulae fruiting body extract, the Pleurotus ferulae mycelium extract and the Pleurotus ferulae mycelium culture reduce skin irritation caused by irritants such as superfactants, flavors and alcohols.

### Example 7

### Preparation of Skin Toner Containing Pleurotus ferulae Fruiting Body Extract

0.05 g of polypyrrolidone, 0.1 g of oleyl alcohol, 0.2 g of polyoxyethylene monooleate, 0.2 g of a flavoring agent, 0.1 g of p-hydroxybenzoic acid methyl ester, a small amount of antioxidant and a small amount of pigment were mixed with and dissolved in 8 g of 95% ethanol. The resulting mixture was added to a solution obtained by dissolving 0.05 g of the Pleurotus ferulae fruiting body extract obtained in Example 1 and 5 g of glycerin in 85.33 g of purified water, followed by stirring, to prepare a skin toner containing the Pleurotus ferulae fruiting body extract.

### Example 8

### Preparation of Skin Toner Containing Pleurotus ferulae Mycelium Extract

0.05 g of polypyrrolidone, 0.1 g of oleyl alcohol, 0.2 g of polyoxyethylene monooleate, 0.2 g of a flavoring, 0.1 g of p-hydroxybenzoic acid methyl ester, a small amount of antioxidant, and a small amount of pigment were mixed with and dissolved in 8 g of 95% ethanol. The resulting mixture was added to a solution obtained by dissolving 0.05 g of the Pleurotus ferulae mycelium extract obtained in Example 2 and 5 g of glycerin in 85.33 g of purified water, followed by stirring, to prepare a skin toner containing the Pleurotus ferulae mycelium extract.

### Example 9

### Preparation of Skin Toner Containing Pleurotus ferulae Mycelium Culture

0.05 g of polypyrrolidone, 0.1 g of oleyl alcohol, 0.2 g of polyoxyethylene monooleate, 0.2 g of a flavoring agent, 0.1 g of p-hydroxybenzoic acid methyl ester, a small amount of antioxidant and a small amount of pigment were mixed with and dissolved in 8 g of 95% ethanol. The resulting mixture was added to a solution obtained by dissolving 0.05 g of the Pleurotus ferulae mycelium culture obtained in Example 3 and 5 g of glycerin in 85.33 g of purified water, followed by stirring, to prepare a skin toner containing the Pleurotus ferulae mycelium culture.

### Example 10

### Preparation of Emulsion Containing Pleurotus ferulae Fruiting Body Extract

1.2 g of cetyl alcohol, 10 g of squalane, 2 g of vaseline, 0.2 g of p-hydroxybenzoic acid ethyl ester, 1 g of glycerin monostearate, 1 g of polyoxyethylene (20 mol-added) monooleate and 0.1 g of a flavoring agent were heated, mixed and dissolved at 70° C., and 0.5 g of the Pleurotus ferulae fruiting body extract obtained in Example 1, 5 g of dipropylene glycol, 2 g of polyethylene glycol-1500, 0.2 g of triethanolamine and 76.2 g of purified water were dissolved by heating 75° C. The two solutions were mixed and emulsified to prepare an oil-in-water (O/W) type emulsion.

### Example 11

### Preparation of Emulsion Containing Pleurotus ferulae Mycelium Extract

1.2 g of cetyl alcohol, 10 g of squalane, 2 g of Vaseline, 0.2 g of p-hydroxybenzoic acid ethyl ester, 1 g of glycerin monostearate, 1 g of polyoxyethylene (20 mol-added) monooleate and 0.1 g of a flavoring agent were heated, mixed and dissolved at 70° C., and 0.5 g of the Pleurotus ferulae mycelium extract obtained in Example 2, 5 g of dipropylene glycol, 2 g of polyethylene glycol-1500, 0.2 g of triethanolamine and 76.2 g of purified water were dissolved by heating to 75° C. The two solutions were mixed and emulsified to prepare an oil-in-water (O/W) type emulsion.

### Example 12

### Preparation of Emulsion Containing Pleurotus ferulae Mycelium Culture

1.2 g of cetyl alcohol, 10 g of squalane, 2 g of vaseline, 0.2 g of p-hydroxybenzoic acid ethyl ester, 1 g of glycerin monostearate, 1 g of polyoxyethylene (20 mol-added) monooleate and 0.1 g of a flavoring agent were heated, mixed and dissolved at 70° C., and 0.5 g of the Pleurotus ferulae mycelium culture obtained in Example 3, 5 g of dipropylene glycol, 2 g of polyethylene glycol-1500, 0.2 g of triethanolamine and 76.2 g of purified water were dissolved by heating 75° C. The two solutions were mixed and emulsified to prepare an oil-in-water (O/W) type emulsion.

### Example 13

### Preparation of Cosmetic Toner Containing Pleurotus ferulae Fruiting Body Extract

5 g of 95% ethyl alcohol, 1.2 g of polyoxyethylene sorbitan monooleate, 0.3 g of chitulose (a complex of chitin and cellulose), 0.2 g of sodium hyaluronate, 0.2 g of vitamin E-acetate, 0.2 g of sodium licorice, 0.1 g of p-hydroxybenzoic acid ethyl ester, 1 g of the Pleurotus ferulae fruiting body extract obtained in Example 1 and a suitable amount of pigment were mixed to prepare a cosmetic toner.

### Example 14

### Preparation of Cosmetic Toner Containing Pleurotus ferulae Mycelium Extract

5 g of 95% ethyl alcohol, 1.2 g of polyoxyethylene sorbitan monooleate, 0.3 g of chitulose, 0.2 g of sodium hyaluronate, 0.2 g of vitamin E-acetate, 0.2 g of sodium licorice, 0.1 g of p-hydroxybenzoic acid ethyl ester, 1 g of the Pleurotus ferulae mycelium extract obtained in Example 2 and a suitable amount of pigment were mixed to prepare a cosmetic toner.

### Example 15

### Preparation of Cosmetic Toner Containing Pleurotus ferulae Mycelium Culture

5 g of 95% ethyl alcohol, 1.2 g of polyoxyethylene sorbitan monooleate, 0.3 g of chitulose, 0.2 g of sodium hyaluronate, 0.2 g of vitamin E-acetate, 0.2 g of sodium licorice, 0.1 g of p-hydroxybenzoic acid ethyl ester, 1 g of the Pleurotus ferulae mycelium culture obtained in Example 3 and a suitable amount of pigment were mixed to prepare a cosmetic toner.

As apparent from the above description, the present invention enables preparation of a high-yield Pleurotus ferulae fruiting body extract or a high-yield Pleurotus ferulae mycelium culture which is multi-functionally effective in providing anti-oxidation, facilitating collagen synthesis, preventing aging, alleviating skin fine wrinkles, whitening skin, moisturizing skin, reducing skin irritation, preventing acne, alleviating atopy, providing anti-inflammation, preventing hair damage, preventing alopecia and facilitating hair growth and preparation of a multifunctional composition for skin external application using the same.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A composition for skin external application comprising a Pleurotus ferulae fruiting body extract as an active ingredient.

2. A composition for skin external application comprising a Pleurotus ferulae mycelium extract as an active ingredient.

3. A composition for skin external application comprising a liquid culture obtained by culturing the Pleurotus ferulae mycelium, as an active ingredient.

4. The composition according to claim 1, wherein the composition comprises the Pleurotus ferulae fruiting body extract, the Pleurotus ferulae mycelium extract or the liquid culture obtained by culturing the Pleurotus ferulae mycelium of an amount of 0.001 to 90.0% by weight, with respect to the weight of the composition.

5. The composition according to claim 1, wherein the composition is a cosmetic composition.

6. The composition according to claim 5, wherein the cosmetic composition is used for whitening skin.

7. The composition according to claim 5, wherein the cosmetic composition is used for preventing aging.

8. The composition according to claim 5, wherein the cosmetic composition is used for alleviating wrinkles.

9. The composition according to claim 5, wherein the cosmetic composition is used for moisturizing skin.

10. The composition according to claim 5, wherein the cosmetic composition is used for reducing skin irritation.

11. The composition according to claim 5, wherein the cosmetic composition is used for alleviating atopic skin.

12. The composition according to claim 5, wherein the cosmetic composition is used for alleviating acne.

13. The composition according to claim 5, wherein the cosmetic composition is used for preventing hair damage.

14. The composition according to claim 5, wherein the cosmetic composition is used for preventing alopecia and improving hair growth.

15. The composition according to claim 1, wherein the composition is a pharmaceutical composition.

16. The composition according to claim 15, wherein the pharmaceutical composition is used for preventing or treating inflammatory diseases.

17. The composition according to claim 15, wherein the pharmaceutical composition is used for preventing or treating atopy.

18. The composition according to claim 15, wherein the pharmaceutical composition is used for preventing or treating acne.
